(19) United States
(12) Patent Application Publication
(10) Pub. No.: US 2009/0203006 A1
(43) Pub. Date: Aug. 13, 2009

(54) BIOLOGICAL MARKERS OF CHRONIC WOUND TISSUE AND METHODS OF USING FOR CRITERIA IN SURGICAL DEBRIDEMENT

(75) Inventors: Harold Brem, Bronx, NY (US); Marjana Tomic-Canic, Hillsdale, NJ (US)

Correspondence Address: DARBY & DARBY P.C., P.O. BOX 770, Church Street Station, New York, NY, 10008-0770, US

(73) Assignee: New York Society for the Ruptured and Crippled Maintaining the Hospital for Special Surgery, New York, NY (US)

(21) Appl. No.: 12/298,118

(22) PCT Filed: May 1, 2007
(86) PCT No.: PCT/US07/10577

§ 371 Date: Oct. 22, 2008

Related U.S. Application Data
(60) Provisional application No. 60/796,902, filed on May 1, 2006.

(51) Int. Cl. C12Q 1/68; C40B 40/08

(52) U.S. Cl. 435/6; 506/17

(57) ABSTRACT

The present invention relates to methods for identifying tissue sites in a chronic wound that are suitable for debridement and whether debridement procedure has been successful using particular biological markers of the cells within the tissue sites of the chronic wounds.

### CROSS REFERENCE TO RELATED APPLICATIONS

The present application is the U.S. National Phase of International Patent Application Serial No. PCT/US07/10577, filed May 1, 2007, which claims the benefit of U.S. Provisional Patent Application Ser. No. 60/796,902, filed May 1, 2006, both of which are hereby incorporated in their entireties.

### FIELD OF INVENTION

The present invention relates to biological markers in cells and tissues from sites in and adjacent to chronic wounds. These markers identify whether cells within a site will respond well to surgical debridement and can be used in methods of determining where to debride a chronic wound and/or when a debridement procedure has been successful.

### BACKGROUND OF THE INVENTION

Chronic ulcers, such as venous ulcers, are characterized by physiological impairments, manifested in delays in healing, which results in severe morbidity. These chronic ulcers are reaching epidemic proportions, mostly affecting the elderly and disabled (Brem et al. (2003) Surg. Tech. Int. 11:161-167). Not only do these chronic ulcers significantly impair an affected person's life, the cost of caring for such chronic wounds is burdensome. Over twenty-five billion dollars was spent in the United States alone on the treatment of chronic wounds, including the costs of surgical debridement, the mainstay of treatment of chronic wounds (Williams et al. (2005) Wound Repair Regen. 13:131-137; Steed et al. (1996) J. Amer. Coll. Surg. 77:575-586).

Accumulation of devitalized tissue, cellular exudates and infection at the outer surface of the wound is characteristic of a chronic wound and prevents adequate cellular response to wound healing stimuli. Wound bed preparation facilitates restoration and regeneration of damaged tissue and provides enhanced function of new therapies (Davies et al. (2005) Brit. J. Nurs. 14:393-97). This wound bed preparation is accomplished by debridement, which is a method of removing devitalized tissue from chronic wounds and decreasing bacterial contamination, while stimulating contraction and epithelialization of the wound (Brem et al. (2004) Amer. J. Surg. 188:1-8). Proper debridement of a chronic ulcer is important for a good clinical outcome. Typically, patients are debrided weekly and it has been shown that sharp debridement increases the healing rate of venous ulcers when compared to the healing rate of non-debrided wounds. Between weeks 8 and 20 post-debridement, 16% of debrided ulcers versus 4.3% of non-debrided ulcers achieved complete healing (Williams et al. (2005) Wound Repair Regen. 13:131-137; Steed et al. (1996) J. Amer. Coll. Surg. 77:575-586). Nevertheless, in contrast to tumor excision and other surgical procedures, objective histological, biological and molecular markers have not been developed for debridement and the procedure remains relatively primitive, as new surgeons are taught to “debride until it bleeds.” Moreover, about 20% of patients never heal. Thus, there is a need to find an objective determinant as to the borders of surgical debridement.

Microarray technology has the ability to simultaneously analyze the expression patterns of the entire genome, thus allowing the identification of pathogenic profiles. Such gene expression profiles of various human tumors have led to the identification of transcriptional patterns related to tumor classification, disease outcome, or response to therapy (Grose (2004) Genome Biol. 5:228; Golub et al. (1999) Science 286:531-37; Risinger et al. (2003) Cancer Res. 63:6-11; and Van de Vijver et al. (2002) New Eng. J. Med. 347:1999-2009). Microarray technology has also been used to study the mechanism of action of specific therapeutics (Wang (2005) Opin. Mol. Ther. 7:246-250) and identify the profile of repair of several tissues, such as cornea, tendons, skin and bone (Cao et al. (2002) Invest. Opthalmol. Vis. Sci. 43:2897-2904; Nakazawa et al. (2004) J. Ortho. Res. 22:520-525; Cole et al. (2001) Wound Repair Regen. 9:360-70). While it has been previously reported that the activation of the β-catenin pathway leads to the induction of c-myc, which contributes to chronic wound development through the inhibition of epithelialization (Stojadinovic et al. (2005) Am. J. Pathol. 167:56-59), the identification of a gene expression profile for the pathogenesis of chronic ulcers remains to be elucidated.

Additionally, therapies other than surgical debridement that stimulate healing of the wound is an essential step in eliminating morbidity caused by the wounds, as well as improving the patients' lives and decreasing healthcare costs. However, there are only two products that are currently approved by the Food and Drug Administration for the treatment of chronic wounds, platelet derived growth factors (Wieman (1998) Amer. J. Surg. 176:74 S-79S) and a cellular therapy called Human Skin Equivalent (Sibbald (1998) J. Cutan. Med. Surg. 3:S1-24-28; Brem et al. (2000) Arch. Surg. 135:627-34). A critical step in development and testing of new therapies is the ability to target responsive cells within the wound that would properly respond to wound healing stimuli.

### SUMMARY OF THE INVENTION

The present invention overcomes the problems in the art by providing markers and methods that identify viable tissue within a wound that has a greater potential to respond to healing stimuli. The present invention also provides methods for determining if a debriding procedure has been successful or if additional debriding treatment is necessary.

The present invention is based upon the surprising discovery that the gene expression profiles of cells and tissues in sites within and adjacent to chronic wounds directly correlate to particular cellular biology and responses. In particular, it has been found that tissue from the site adjacent to a chronic wound (for convenience, herein referred to as “ACW”) contains cells with a morphology similar to that of healthy cells, an increased capacity to migrate, and good response to wound healing stimuli. The tissue from sites within the wound, such as the non-healing edge of the wound (hereinafter referred to as “NHE”), contains cells that exhibit pathological morphology, a decreased ability to migrate, and poor response to wound healing stimuli. More importantly, the tissues from these two sites possess distinct gene expression profiles. Thus, these gene expression profiles provide a convenient marker for determining which tissue is suitable for debriding as well as whether a debriding procedure has been successful.

Additionally it has been found that certain genes are induced or suppressed in the cells in the tissues in the specific wound sites. Thus, these genes can be used as markers for further determining the metes and bounds of a debridement procedure.

One embodiment of the present invention provides for a method for the identification of a margin of debridement within or adjacent to a chronic wound, by (a) obtaining a tissue sample from a site within or adjacent to the chronic wound; (b) determining a gene expression profile of the tissue sample; and (c) comparing the gene expression profile of the tissue sample with a known gene expression profile of tissue from a known site adjacent to the chronic wound (ACW). If the gene expression profile of the tissue sample, such as from the NHE, is the same or similar to the known gene expression profile of the tissue from the known site, such as the ACW, then the site of the tissue sample is within the margin of debridement (i.e., debrided sufficiently).

A preferred embodiment of this method of the invention is that the tissue from the known site contains cells with healthy, normal morphology that respond well to wound healing stimuli. A further preferred embodiment of this method would be that the tissue from the known site be from the non-ulcerated skin adjacent to the chronic wound.

It is also preferred that the gene expression profile for both the tissue sample (NHE) and the tissue from the known site be determined by microarray analysis. The known site is preferably from the ACW. The gene expression profile of the tissue from the known site could be determined prior to performing the method of the invention. After this gene expression profile of the tissue of the known site is determined, it can be used for comparison in performing the method of the invention once or several subsequent times.

It is also contemplated that the gene expression profile of the tissue sample be compared to the known gene expression profile for non-ulcerated skin adjacent to the chronic wound (ACW) as set forth in FIG. 2. If the gene expression profile of the tissue sample is the same or similar to the known gene expression profile, then the site is within the margin of debridement (i.e., debrided sufficiently).

In a further embodiment of this method, particular genes, i.e., “marker genes,” are either induced or suppressed in the cells in the tissue from the known site, such as the ACW or normal healthy skin away from the wound. These marker genes for the tissue from the known site can also be determined by microarray analysis. A comparison of the expression of genes by cells in the tissue sample, such as from the NHE, to the expression of the marker genes in the cells of the known site can then also be used to determine if the site of the tissue sample is suitable for debriding.

This method can be used in a clinical setting to determine where in a wound a debridement procedure should commence, as well as determine the margin of debridement. This method can also be used to identify sites in and adjacent to a wound that would respond well to other therapeutic agents that are being used or tested to further treat the chronic wound.

Another embodiment of the invention provides for a method for determining whether a chronic wound is in further need of debridement, by (a) obtaining a tissue sample from within the chronic wound (NHE); (b) determining a gene expression profile for the tissue sample; (c) comparing the gene expression profile of the tissue sample with a known gene expression profile of tissue from a known site adjacent to the chronic wound. If the gene expression profile of the tissue sample is the same or similar to the known gene expression profile of the tissue from the known site adjacent to the wound (ACW), then the wound is not in need of further debridement. If the gene expression profile of the tissue sample is not the same or similar to the known gene expression profile of the tissue from the known site adjacent to the wound (ACW), then the debriding procedure should continue until the known gene expression profile is obtained.

Again a preferred embodiment of this method of the invention is that the tissue from the known site contains cells with healthy, normal morphology that respond well to wound healing stimuli.

It is also preferred that the gene expression profile for both the tissue sample (NHE) and the tissue from the known site, such as ACW, be determined by microarray analysis. The gene expression profile of the tissue from the known site could be determined prior to performing the method of the invention. After the gene expression profile of the tissue of the known site is determined, it can be used for comparison in performing the method of the invention once or several subsequent times.

It is also contemplated that the gene expression profile of the tissue sample be compared to the known gene expression profile for the non-ulcerated skin adjacent to a chronic wound (ACW) as set forth in FIG. 2. If the gene expression profile of the tissue sample is the same or similar to the known gene expression profile, then debridement has been successful. It is also preferred but not necessary that the sample tissue come from a site that has been previously debrided.

In a further embodiment of this method, particular genes, i.e., “marker genes,” are either induced or suppressed in the cells in the tissue from the known site, either the ACW or normal healthy skin. These marker genes for the tissue from the known site can also be determined by microarray analysis. A comparison of the expression of genes by cells in the tissue sample, such as from the NHE, to the expression of the marker genes in the cells of the known site can then also be used to determine if debridement has been successful.

This method can be used in a clinical setting to determine if a wound has been successfully debrided. This method can also be used to identify sites in a chronic wound that because it has been successfully debrided would now respond well to other therapeutic agents that are being used or tested to further treat the chronic wound.

A further embodiment of the invention is the gene expression profile of the non-ulcerated skin adjacent to a chronic wound (ACW) as set forth in FIG. 2, the gene expression profile of normal healthy skin as set forth in FIG. 7, and the gene expression profile of the non-healing edge of a chronic wound (NHE) as set forth in both FIGS. 2 and 7. Such expression profiles are convenient and useful markers for comparing the gene profile expression of tissue samples in and adjacent to a chronic wound to determine if the tissue is suitable for debridement, if it is within the margin of debridement and/or if debriding has been successful

### BRIEF DESCRIPTION OF THE DRAWINGS

FIG. 1 shows that distinct wound locations have specific histology. FIG. 1(a) depicts a typical venous stasis ulcer. Arrows point to the regions from which tissue biopsies were obtained. Location A is the non-healing edge of the ulcer (NHE) and location B is the adjacent, non-ulcerated skin (ACW). FIG. 1(b) depicts hematoxylin and eosin stained biopsies of epidermis from the non-healing edge (location A), the adjacent, non-ulcerated skin (location B), and normal skin. FIG. 1(c) depicts hematoxylin and eosin stained biopsies of dermis from the non-healing edge (location A), the adjacent, non-ulcerated skin (location B), and normal skin. FIG. 1(d) depicts the staining of the biopsies from the non-healing edge (location A), the adjacent, non-ulcerated skin (location B), and normal skin with pro-collagen. Circles demarcate the location from which the enlarged images are shown in the insets below. The scale bar is 100 μm.

FIG. 2 depicts the distinct gene expression patterns for the tissues from the different wound locations, the non-healing edge (NHE) (location A) and the adjacent, non-ulcerated skin (ACW) (location B).

FIG. 3 shows fibroblast cells grown from the tissue from the non-healing edge (NHE) (location A) and the adjacent, non-ulcerated skin (ACW) (location B).

FIG. 4 shows the results of an in vitro wound scratch assay. FIG. 4(a) depicts the actual experiment with the full lines indicating the initial wound area and the dotted line demarcating the migrating front of the cells. FIG. 4(b) depicts a graph showing the average coverage of the scratch wound widths in percent (%) relative to baseline wound at 0, 4, 8 and 24 hours for each cell type.

FIG. 5 shows the gene expression profiles for tissues obtained from three wound locations: location A, the non-healing edge of the wound (NHE); location B, the adjacent, non-ulcerated skin (ACW); and location *, an intermediate location between location A and location B.

FIG. 6 shows the gene annotation table describing the molecular function and biological categories of the genes present on the Affymetrix Human Genome U133 GeneChip®. The light gray areas depict genes that are up-regulated in the tissue at location B, the non-ulcerated skin adjacent to the chronic wound (ACW), as compared to the tissue at location A, the non-healing edge of the wound (NHE). The dark gray areas depict genes that are down-regulated in tissues from location B as compared to location A. The numbers within the light and dark gray shaded areas depict the fold change. The two different columns depict the comparison of the two locations in two different patients.

FIG. 7 depicts the distinct gene expression patterns for the tissues from the two different skin samples, chronic non-healing wounds, and normal healthy skin.

FIG. 8 depicts the 100 most differentially regulated genes between skin from chronic non-healing wounds and normal healthy skin. Fifty (50) of the genes are up-regulated in skin from chronic non-healing wounds as compared to normal skin, and fifty (50) are down-regulated. The genes are grouped by cellular functions and biological processes. Associated fold changes and p-values are also presented.

FIG. 9 shows the results of immunohistochemistry analysis of normal healthy skin and skin from the non-healing edge of a chronic wound stained with antibodies that recognize desmoglein 2, desmoglein 3, and desmoplakin.

FIG. 10 shows the results of immunohistochemistry analysis of normal healthy skin and skin from the non-healing edge of a chronic wound stained with antibodies that recognize involucrin, keratin 10, and filaggrin.

FIG. 11 depicts the results of RT-PCR using tissue from non-healing chronic wounds and normal healthy skin. FIG. 11(A) shows results for the measurement of expression of genes MMP11, S100A7, and DEFB4. FIG. 11(B) shows the results for the measurement of the expression of genes BMP2, BMP7, and KLK6. FIG. 11(C) shows the results for the measurement of the expression of genes APOD and CCL27.

### DETAILED DESCRIPTION OF THE INVENTION

There are presently no objective indicia to serve as a guide in surgical debridement, either as to which portion of a chronic wound should be debrided or as to how a wound is responding to debriding treatment. The present invention sets forth criteria and methods for determining both.

To assess the pathogenic state of wound tissue before and after wound debridement, biopsies from distinct locations in a chronic wound were analyzed as to their histology, biology and gene expression profile. It was found that biopsies from the non-healing edges of a wound have a specific identifiable and reproducible gene expression profile and primary fibroblasts deriving from this site have impaired migration capacity. In contrast, biopsies from the adjacent non-ulcerated locations of the wound have a different specific gene expression profile and the primary fibroblasts deriving from this location have a similar migration capacity as normal primary fibroblasts. Thus, chronic ulcers contain distinct sub-populations of cells with different capacities to heal and gene expression profiling can be used to identify them.

### DEFINITIONS

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in this specification, including examples of any terms discussed herein, is illustrative only, and in no way limits the scope and meaning of the invention or any exemplified term. Likewise, the invention is not limited to its preferred embodiments.

The term “adjacent” refers to a location near or close to a chronic wound edge that may or may not be in actual contact with the wound.

The terms “expression profile” or “gene expression profile” are used interchangeably and refer to any description or measurement of one of more of the genes that are expressed by a cell, tissue, or organism under or in response to a particular condition. Expression profiles can identify genes that are up-regulated, down-regulated, or unaffected under particular conditions. Gene expression can be detected at the nucleic acid level or at the protein level. The expression profiling at the nucleic acid level can be accomplished using any available technology to measure gene transcript levels. For example, the method could employ in situ hybridization, Northern hybridization or hybridization to a nucleic acid microarray, such an oligonucleotide microarray, or a cDNA microarray. Alternatively, the method could employ reverse transcriptase-polymerase chain reaction (RT-PCR). The expression profiling at the protein level can be accomplished by any available technology to measure protein levels, e.g., using peptide-specific capture agent arrays (see, e.g., International PCT Publication No. WO 00/04389).

The term “same” as used herein as related to gene expression profiles from cells of a tissue means that upon visual examination alone, the gene expression profiles appear identical.

The term “similar” as used herein as related to gene expression profiles from cells of a tissue means that upon visual examination alone, the gene expression profiles appear nearly but not exactly identical.

The phrase “identical or similar expression” (and the like) as used herein refers to an expression level of a gene or product thereof (i.e., an mRNA transcript or protein) in a tissue sample that is ±30%, preferably ±20%, and more preferably ±10% of a given numerical value of the expression level of the same gene or gene product from the tissue of a known site as determined by any quantitative assay known in the art.

The term “margin of debridement” as used herein means an area of skin at the non-healing edge that contains tissue that is biologically responsive to wound healing stimuli and where the debridement procedure should end.

The term “agent” is used herein to mean a substance capable of producing a chemical reaction or a physical or a biological effect. An agent could be, among other things, a chemical, including a nucleic acid; a drug; a virus; or a bacterium.

Cells from different regions of a chronic wound exhibit different cell morphology. Cells derived from tissue from the non-healing edge of a wound (NHE) exhibited pathological morphology whereas cells derived from tissue adjacent to the wound (ACW) exhibited normalized pathology.

Additionally, cells from different specific regions of a chronic wound exhibit unique characteristics, such as cell migration and cellular response to wounding, that would influence the success of debridement treatment, since the aim of debridement of a wound is not only to clean the necrotic tissue but to reach out to the cells within the wound that are biologically capable of responding to wound healing stimuli. Cells grown from tissue obtained from the non-healing edge of a chronic wound (NHE) show a diminished capacity to migrate and respond to wounding, whereas cells derived from tissue from the adjacent, non-ulcerated area of the chronic wound (ACW) show an increased capacity to migrate and respond well to wound healing stimuli. Typically, this area adjacent to the ulcer is the margin where debridement ends. However, based upon the ability of the cells in this area to migrate and heal, this area should be included in the debridement treatment since the time of healing could be reduced if more permissive cells were exposed to wound healing signals. Moreover, these cells with the greater ability to respond to wound healing stimuli would also be a preferred target for other therapeutic treatment for a chronic wound, such a pharmaceutical or biological agent.

Perhaps, more surprising is that these cells from different regions of the chronic wound are not only characterized by unique biological properties, but are also characterized by a unique gene expression profile. Gene expression profiles resemble a bar code and allow overall visualization of an entire expression pattern rather than specific gene regulation. Since there is a direct correlation between biological properties that may be useful determining criteria for debridement and a unique gene expression profile in cells from different regions of a chronic wound, gene expression profiling can serve as a guide for surgical debridement in the treatment of chronic ulcers. The differences in the gene expression maps of the particular wound locations are definitive and can be grouped as specific patterns that can be used as a diagnostic tool.

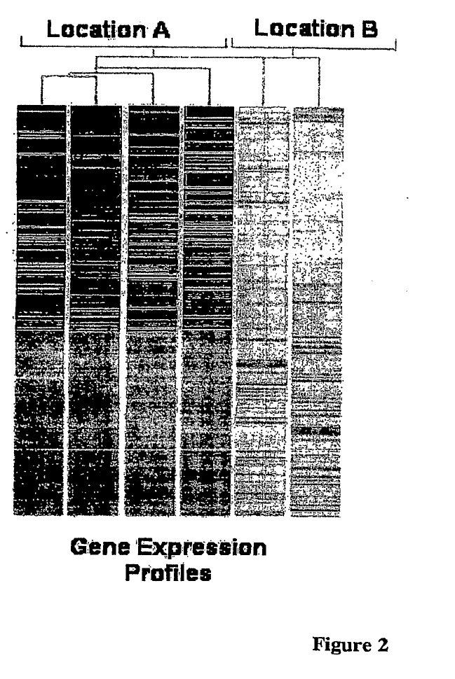

As shown in FIG. 2, the gene expression profiles or patterns from tissues in the non-healing edge of a wound (NHE) are the same or similar to each other but markedly different from the gene profiles of the tissues in the non-ulcerated skin adjacent to the wound (ACW). These profiles resemble bar codes with the dark gray lines representing up-regulated genes, the lighter gray lines representing down-regulated genes, and the lightest gray lines representing the expressed genes. By referring to the gene expression profiles set forth FIG. 2, it can be seen that the gene expression profiles of the tissue from the non-ulcerated skin adjacent to the wound comprises mostly lightest gray lines in its pattern whereas the gene expression profiles of the tissues from the non-healing edge of the wound are mostly dark gray on top and lighter gray on the bottom. Thus, the cells in the tissue in the non-healing edge of the chronic wound (NHE) either up- or down-regulate many genes that are expressed in the cells of the non-ulcerated skin adjacent to the wound (ACW).

As shown in FIG. 7, the gene expression profiles or patterns from tissues in the non-healing edge of a wound (NHE) are similar to each other and the profiles for chronic wounds in FIG. 2. The gene expression profiles or patterns for the healthy control skin away from the chronic wound is also markedly different from the gene profiles of the skin from the chronic wounds. Referring to FIG. 7, it can be seen that the gene expression profiles of the tissue from the chronic wounds comprise dark gray and lighter gray lines at opposite areas in the pattern as compared to the profiles for the healthy skin. Thus, the cells in the tissues of the chronic non-healing wounds differentially regulate genes as compared to healthy skin.

The similarity of the patterns of the gene expression profiles from tissue derived from the same location (either the NHE or the ACW or healthy skin), and the differences in the patterns of the gene expression profiles of the different types of tissue are easily visually discernable by the naked eye. Thus, by generating a gene expression profile of the specific wound region, one could quickly identify, by visual examination only, from which region a tissue biopsy originates and determine if it contains cells which would respond well to debridement as well as determine how well the wound has been debrided.

It is also possible to quantify the data in the gene expression profiles and determine which genes in particular are being up-regulated, i.e., induced, or down-regulated, i.e., suppressed, in the tissues from the different locations. Table 1 lists genes that are up-regulated in the non-ulcerated skin adjacent to the wound (ACW) (in alphabetical order as to function) relative to the genes in the non-healing edge of the wound (NHE) and Table 2 lists the genes that are down-regulated in the non-ulcerated skin adjacent to the wound (ACW) (in alphabetical order as to function) relative to the genes in the non-healing edge of the wound (NHE). Thus, the specific regulation of any one gene or combination of genes in a tissue sample or biopsy can be determined and compared to the regulation of genes in the non-ulcerated skin adjacent to the wound. This comparison of the regulated genes in the tissue sample to the regulation of any of the marker genes in the non-ulcerated skin adjacent to the wound can assist in further determining if the tissue sample contains cells which will respond well to debridement and/or how well a wound has been debrided.

TABLE 1
Genes which are up-regulated or induced in the non-ulcerated skin adjacent to a
wound as compared to the non-healing edge of a wound

| Function | Gene |
|---|---|
| Adhesion | tenascin C (hexabrachion) |
| Adhesion | desmocollin 2 |
| Adhesion | CD47 antigen (Rh-related antigen, integrin- |
| | associated signal transducer) |
| Adhesion | melanoma cell adhesion molecule |
| Adhesion | carcinoembryonic antigen-related cell adhesion |
| | molecule 6 |
| Adhesion | caldesmon 1 |
| Anti-oxidant | glutathione S-transferase omega 1 |
| Apoptosis | tumor necrosis factor receptor superfamily, |
| | member 21 |
| Apoptosis inhibitor | immediate early response 3 |
| Ca binding | EGF-like domain, multiple 6 |
| Ca binding | reticulocalbin 3, EF-hand calcium binding |
| | domain |
| Ca binding | calumenin |
| Cell cycle | CDC20 cell division cycle 20 homolog (S. cerevisiae) |
| Cell cycle | CDC28 protein kinase regulatory subunit 2 |
| Cell cycle | ZW10 interactor |
| Cell cycle | regulator of G-protein signaling 2, 24 kDa |
| Cell cycle | cell division cycle 25 B |
| Cell cycle inhibitor | quiescin Q6 |
| Cell growth proliferation | cysteine-rich, angiogenic inducer 61 |
| Cytoskeletal | thymosin, beta 10 |
| Cytoskeletal | transgelin |
| Cytoskeletal, actin | tropomyosin 2 (beta) |
| Cytoskeletal, actin | actin related protein 2/3 complex, subunit 1B, 41 kDa |
| Cytoskeletal, actin | actinin, alpha 1 |
| Cytoskeletal, actin | erythrocyte membrane protein band 4.1-like 3 |
| Cytoskeletal, actin | actin, alpha 2, smooth muscle, aorta |
| Cytoskeletal, actin | actin, beta |
| Cytoskeletal, keratin | keratin 17 |
| Cytoskeletal, keratin | keratin 16 |
| Cytoskeletal, keratin | cytokeratin type II |
| Cytoskeletal, keratin | keratin 6A |
| Cytoskeletal, myosin | myosin, heavy polypeptide 10, non-muscle |
| Cytoskeletal, tubulin | tubulin, beta 4 |
| Cytoskeletal, tubulin | tubulin, alpha, ubiquitous |
| Cytoskeletal, tubulin | tubulin, beta MGC4083 |
| Cytoskeletal, tubulin | tubulin, alpha 6 |
| Cytoskeletal, tubulin | tubulin, beta 5 |
| Cytoskeletal, tubulin | tubulin, alpha 3 |
| Cytoskeletal, tubulin | tubulin beta 2 |
| DNA binding, histone | H2A histone family, member X |
| DNA binding, histone | H2A histone family, member Z |
| DNA repair, synthesis | ribonucleotide reductase M2 polypeptide |
| DNA repair, synthesis | uridine phosphorylase 1 |
| DNA repair, synthesis | cytidine deaminase |
| ECM | fibronectin 1 |
| ECM | spondin 2, extracellular matrix protein |
| ECM | collagen, type XI, alpha 1 |
| ECM | collagen, type V, alpha 3 |
| ECM | thrombospondin 1 |
| ECM | syndecan 2 |
| ECM | collagen, type IV, alpha 2 |
| ECM | biglycan |
| ECM | fibronectin 1 |
| Energy | lactate dehydrogenase B |
| Energy | aldo-keto reductase family 1, member B1 |
| Enzyme | transketolase (Wernicke-Korsakoff syndrome) |
| Epidermal differentiation | S100 calcium binding protein A2 |
| Epidermal differentiation | S100 calcium binding protein A6 (calcyclin) |
| Epidermal differentiation | small proline-rich protein 2B |
| Epidermal differentiation | small proline-rich protein 1A |
| Epidermal differentiation | S100 calcium binding protein A11 (calgizzarin) |
| Epidermal differentiation | S100 calcium binding protein A10 (annexin II |
| | ligand, calpactin I, light polypeptide (p11) |
| Epidermal differentiation | S100 calcium binding protein A4 (calcium |
| | protein, calvasculin, metastasin, murine placental homolog) |
| Golgi apparatus | coatomer protein complex, subunit zeta 2 |
| Hemoglobin | hemoglobin, gamma G |
| Immunoglobulin | immunoglobulin kappa variable ID-13 |
| Interferon-regulated | interferon, alpha-inducible protein |
| Membrane protein | tyrosine 3-monooxygenase/tryptophan 5- |
| | monooxygenase activation protein, theta |
| | polypeptide |
| Membrane protein | Thy-1 cell surface antigen |
| Membrane protein | transmembrane 4 superfamily member 1 |
| Membrane protein | CD151 antigen |
| Metabolism, amino acid | lysyl oxidase-like 2 |
| Metabolism, amino acid | lysyl oxidase-like 1 |
| Metabolism | ornithine decarboxylase 1 |
| Metabolism, steroid | aldo-keto reductase family 1, member C1 |
| Metabolism, steroid | aldo-keto reductase family 1, member C2 |
| Mitochondrial | cytochrome c oxidase subunit VIIa polypeptide |
| | 1 (muscle) |
| Nuclear receptor | nucleophosmin |
| Nucleoskeletal | karyopherin alpha 2 |
| Oncogenesis | four and half LIM domains 2 |
| Phosphatase | endoglin (Osler-Rendu-Weber Syndrome 1) |
| Proteolysis | calpain 2, (m/II) large subunt |
| Proteolysis | protease, serine, 23 |
| Proteolysis | WAP four-disulfide core domain 1 |
| Proteolysis | cathepsin L |
| Proteolysis inhibitor | serine (or cysteine) proteinase inhibitor, clade B, |
| | member 13 |
| Proteolysis inhibitor | cystatin B |
| Proteolysis inhibitor | secretory leukocyte protease inhibitor |
| Proteolysis inhibitor | protease inhibitor 3, skin-derived |
| Proteolysis inhibitor | serine (or cysteine) proteinase inhibitor, clade B, |
| | member 1 |
| Proteolysis inhibitor | serine (or cysteine) proteinase inhibitor, clade H, |
| | member 1 |
| Proteolysis inhibitor | tissue inhibitor of metalloproteinase 1 |
| Proteolysis, extracellular | kallikrein 12 |
| Proteolysis, ubiquitin | ubiquitin-conjugating enzyme E2C |
| Proteolysis, ubiquitin | ubiquitin-conjugating enzyme E2S |
| Receptor | low density lipoprotein receptor |
| Receptor | angiotensin II receptor-like 1 |
| Regulator | annexin A1 |
| Regulator | guanylate cyclase 1, soluble, alpha 3 |
| Regulator | annexin A11 |
| Regulator | CAP 1 |
| Regulator | annexin A6 |
| Regulator | annexin A5 |
| Regulator | SH3 domain binding glutamic acid-rich protein- |
| | like 3 |
| Regulator | RAB 31 |
| Secreted | lectin, galactoside-binding, soluble 1 |
| Secreted | latent transforming growth factor beta binding |
| | protein 1 |
| Secreted | insulin-like growth factor binding protein 2 |
| Secreted | insulin-like growth factor binding protein 6 |
| Secreted | chemokine (C-C motif) ligand 18 |
| Secreted | transforming growth factor, beta induced, 68 kDa |
| Secreted | endothelial cell growth factor 1 (platelet- |
| | derived) |
| Secreted | angiopoietin-like 2 |
| Trafficking, vesicles | KDEL endoplasmic reticulum protein retention |
| | receptor 3 |
| Trafficking, vesicles | plasmalemma vesicle associated protein |
| Transcription | pituitary tumor-transforming 1 |
| Transcription | polymerase (RNA) II polypeptide L, 7.6 kDa |
| Transcription factor | cysteine-rich protein 1 |
| Transcription repressor | inhibitor of DNA binding 3, dominant negative |
| | helix-loop-helix protein |
| Transcription repressor | eukaryotic translation initiation factor 4E |
| | binding protein 1 |
| Translation | ribosomal protein S26 |
| Translation | FXYD domain containing ion transport |
| | regulator 5 |
| Transporter, channel | NEL-like 2 |
| Transporter | chloride intracellular channel 3 |
| Tumor suppressor | serologically defined colon cancer antigen 33 |
| Tumor antigen | melanoma associated gene |
| Unknown | Raft-linking protein |
| Unknown | Calcium regulated heat stable protein 1, 24 kDa |
| Unknown | DKFZP586L151 protein |
| Unknown | Hematological and neurological expressed 1 |
| Unknown | Ring finger protein 141 |
| Unknown | Proteoglycan 1, secretory granule |
| Unknown/hypothetical | hypothetical protein PRO1855 |
| Unknown/hypothetical | hypothetical protein FLJ23221 |

TABLE 2
Genes which are down-regulated or suppressed in the non-ulcerated
skin adjacent to a wound as compared to the non-healing edge of a wound

| Function | Gene |
|---|---|
| Adhesion | calsytenin 1 |
| Adhesion | discs, large homolog (Drosophila) |
| Adhesion | protocadherin 21 |
| Adhesion | FAT tumor suppressor homolog 2 (Drosophila) |
| Adhesion | catenin, delta 1 |
| Adhesion | cadherin, EGF LAG seven-ass G-type receptor 2 |
| Adhesion | desmocollin 1 |
| Adhesion | bullous pemphigold antigen 1, 230/240 kDa |
| Adhesion | gap junction protein, beta 3, 31 kDa |
| Antioxidant | glutathione S-transferase A4 |
| Antioxidant | selenoprotein P, plasma, 1 |
| Antioxidant | microsomal glutathione S-tranferase 2 |
| Antioxidant | glutaredoxin (thioltransferase) |
| Antioxidant | catalase |
| Apoptosis | p8 protein |
| Apoptosis | programmed cell death 4 |
| Apoptosis | PRKC, apoptosis, WT1 regulator |
| Apoptosis inhibitor | secreted frizzle-related protein |
| Apoptosis inhibitor | sema domain, immunoglobulin domain (Ig), transmembrane |
| | domain and short cytoplasmic domain |
| Ca binding | signal peptide, CUB domain, EGF-like 2 |
| Cell cycle | cullin 3 |
| Cell cycle | transforming, acidic coil containing protein 2 |
| Cell cycle inhibitor | sestrin 1 |
| Cell cycle inhibitor | B-cell translocation gene 1, anti-proliferative |
| Cell cycle inhibitor | BTG family, member 2 |
| Cell cycle inhibitor | growth arrest-specific 7 |
| Cell growth proliferation | four and a half LIM domains 1 |
| Cytoskeletal | supervilllin |
| Cytoskeletal, actin | spectrin, beta, non-erythrocytic 5 |
| Cytoskeletal, actin | GABA(A) receptor associated protein-like 2 |
| Cytoskeletal, actin | Huntington interacting protein-1 related |
| Cytoskeletal, keratin | keratin 15 |
| Cytoskeletal, keratin | keratin 2A |
| Cytoskeletal, keratin | keratin 23 |
| Cytoskeletal, keratin | keratin 9 |
| Cytoskeletal, keratin | keratin 10 |
| Cytoskeletal, keratin | keratin 1 |
| Cytoskeletal, membrane | uroplakin 1A |
| Cytoskeletal, motility | dynein, cytoplasmic, light polypeptide 2A |
| Cytoskeletal, myosin | myosin X |
| Cytoskeletal, Rho, CDC42 | PTPL1-associated RhoGAP 1 |
| Cytoskeletal, Rho, CDC42 | CDC42 effector protein |
| Cytoskeletal, Rho, CDC42 | T-cell lymphoma invasion and metastasis 1 |
| Cytoskeletal, Rho, CDC42 | Rho guanine nucleotide exchange factor (GEF) 5 |
| Cytoskeletal, tubulin | micro-tubule-associated protein 1 light chain 3 beta |
| Detoxification | paraoxonase 2 |
| Detoxification | monoamine oxidase A |
| Detoxification | flavin containing monooxygenase 2 |
| DNA repair, synthesis | deoxyribonuclease I-like 2 |
| DNA repair, synthesis | cell death-inducing DFFA effector |
| DNA repair, synthesis | adenylate kinase 3 |
| DNA repair, synthesis | DNA-damage-inducible transcript 4 |
| ECM | tuftelin 1 |
| ECM | microfibrillar-associated protein 4 |
| ECM | chitinase 3-like 2 |
| ECM | cartilage oligomeric matrix protein |
| ECM | chondroitin sulfate proteoglycan 2 |
| ECM | fibulin 2 |
| ECM | dermatopontin |
| Energy | aldolase C, fructose-biphosphate |
| Energy | thioredoxin interacting protein |
| Energy | aldehyde dehydrogenase 3 family, member A1 |
| Energy | aldehyde dehydrogenase 4 family, member A1 |
| Energy | aldehyde dehydrogenase 3 family, member B2 |
| Enzyme | P450 (cytochrome) oxidoreductase |
| Epidermal differentiation | small proline-rich protein 3 |
| Epidermal differentiation | S100 calcium binding protein A12 |
| Epidermal differentiation | S100 calcium binding protein A13 |
| Epidermal differentiation | calmodulin-like 5 |
| Epidermal differentiation | ARS component B |
| Epidermal differentiation | small proline rich-like (epidermal differentiation complex) 1B |
| Epidermal differentiation | psoriasis susceptibility 1 candidate 2 |
| Epidermal differentiation | annexin A9 |
| Epidermal differentiation | loricrin |
| Epidermal differentiation | filaggrin |
| Epidermal differentiation | transglutaminase 3 |
| Epidermal differentiation | sciellin |
| Golgi apparatus | bicaudal D homolog 2 (drosophila) |
| Golgi apparatus | golgi auto antigen, golgin subfamily a, 7 |
| Golgi apparatus | DNA segment on chromosome 4, 234 expressed sequence |
| G-regulated protein | ADP-ribosylation factor-like 4 |
| G-regulated protein | ADP-ribosylation factor-like 5 |
| G-regulated protein | ADP-ribosylation factor-like 10C |
| G-regulated protein | ral guanine nucleotide dissociation stimulator |
| Heat shock, chaperone | heat shock 70 kDa protein 2 |
| Heat shock, chaperone | heat shock 70 kDa protein 1A |
| Immune response | D component of complement |
| Immune response | major histocompatibility complex, class I, F |
| Immune response | major histocompatibility complex, class I, A |
| Immune response | major histocompatibility complex, class I, C |
| Immune response | major histocompatibility complex, class II, DR |
| | beta 4 |
| Immunoglobulin | Fc fragment of IgG binding protein |
| Immunoglobulin | immunoglobulin superfamily, member 3 |
| Immunoglobulin | lymphocyte antigen 6 complex, locus G6C |
| Interferon regulated | guanylate binding protein 2, interferon inducible |
| Melanogenesis | tyrosinase-related protein I |
| Melanogenesis | tyrosinase (oculocutaneous albinism 1A) |
| Melonogenesis | dopochrome tautomerase |
| Membrane protein | epithelial membrane protein 2 |
| Membrane protein | melan-A |
| Membrane protein | perixisomal membrane protein 4, 24 kDa |
| Membrane protein | glycoprotein (transmembrane) NMB |
| Membrane protein | transmembrane 7 superfamily member 2 |
| Membrane protein | adipose differentiation-related protein |
| Membrane protein | KIAA0247 |
| Membrane protein | sema domain, immunoglobulin domain |
| | transmembrane domain, short cytoplasmic |
| | domain (semaphorin) 4C |
| Membrane protein | membrane interacting protein of RGS16 |
| Metabolism, amino acid | histidine ammonia-lyase |
| Metabolism, amino acid | arginase, liver |
| Metabolism, amino acid | autism susceptibility candidate 2 |
| Metabolism, amino acid | ornthine aminotransferase (gyrate atrophy) |
| Metabolism, amino acid | phosphoglycerate dehydrogenase |
| Metabolism, carbohydrate | sorbitol dehydrogenase |
| Metabolism, lipid | degenerative spermatocyte homolog, lipid |
| | desaturase (Drosophila) |
| Metabolism, lipid | acyl-CoA synthetase long-chain family member 1 |
| Metabolism, lipid | phosphatidic acid phosphatase type 2B |
| Metabolism, lipid | phospholipid transfer protein |
| Metabolism, lipid | phospholipase A2, group IVB (cytosolic) |
| Metabolism, other | transcobalamin I |
| Metabolism, other | arylsulfatase F |
| Metabolism, other | arylacetamide deacetylase (esterase) |
| Metabolism, other | lactotransferrin |
| Metabolism, other | carbonic anhydrase XII |
| Metabolism, other | anhydrolase domain containing 9 |
| Metabolism, other | spermine oxidase |
| Metabolism, other | glycine amidinotransferase |
| Metabolism, steroid | 24-dehydrocholesterol reductase |
| Metabolism, steroid | START domain containing 5 |
| Metabolism, steroid | oxysterol binding protein-like 8 |
| Mitochondrial | PET112-like yeast |
| Nuclear receptor/RA | RAR-related orphan receptor A |
| Nuclear receptor/RA | retinoid X receptor, alpha |
| Phosphatase | acid phosphatase, prostate |
| Phosphatase | protein phosphatase 3, catalytic subunit, alpha |
| | isoform |
| Phosphatase | dual specificity phosphatase 1 |
| Phosphatase | protein phosphatase 2, regulatory subunit B, |
| | alpha |
| Protein binding | KIAA0795 protein |
| Protein kinase | casein kinase 2, alpha prime polypeptide |
| Protein kinase | SFRS protein kinase 1 |
| Protein kinase | casein kinase 2, beta polypeptide |
| Protein kinase | serum/glucocortoid regulated kinase |
| Protein kinase | MAP kinase-interacting serine/threonine kinase 2 |
| Protein kinase | protein kinase C and casein kinase substrate in neurons 2 |
| Protein kinase inhibitor | protein kinase, lysine deficient 1 |
| Protein modification | phosphatidylinositol glycan, class C |
| Proteolysis | insulin-degrading enzyme |
| Proteolysis | cathepsin L2 |
| Proteolysis | bleomycin hydrolase |
| Proteolysis | calpain 3 |
| Proteolysis | cathepsin H |
| Proteolysis | carboxypeptidase A4 |
| Proteolysis | cathepsin D |
| Proteolysis | protein x 0001 |
| Proteolysis inhibitor | cystatin E/M |
| Proteolysis inhibitor | serine (or cysteine) proteinase inhibitor, clade B, |
| | member 7 |
| Proteolysis inhibitor | serine (or cysteine) proteinase inhibitor, clade B, |
| | member 8 |
| Proteolysis, extracellular | protease, serine, 8 |
| Proteolysis, extracellular | serine protease inhibitor, Kunitz type 1 |
| Proteolysis, ubiquitin | F-box and WD-40 domain protein 7 |
| Receptor | Coxsackie virus and adenovirus receptor |
| Receptor | CD36 antigen |
| Receptor | discoidin domain receptor family, member 1 |
| Receptor | insulin receptor substrate 2 |
| Receptor | putative chemokine receptor |
| Receptor | EphB6 |
| Receptor | G protein-coupled receptor 87 |
| Receptor | fibroblast growth factor receptor 2 |
| Receptor | fibroblast growth factor receptor 3 |
| Receptor | activin A receptor, type IB |
| Receptor | v-erb-b2 erythroblastic leukemia viral oncogene |
| | homolog 2, neuron/glioblastoma derived |
| | oncogene homolog (avian) |
| Receptor | epidermal growth factor receptor (erythroblastic |
| | leukemia viral (v-erb-b) oncogene homolog |
| | (avian) |
| Receptor | protein tyrosine phosphatase, receptor type F |
| Regulator | annexin A4 |
| Regulator | SH3 domain containing Ysc84-like 1 (s. cerevisiae) |
| Regulator | SH3 domain binding glutamic acid rich protein |
| | like |
| Regulator | vav 3 oncogene |
| Regulator | glucosidase, beta: acid |
| Regulator | sphingomyelin phosphodiesterase acid-like 3A |
| Regulator | sphingomyelin phosphodiesterase 1 acid |
| | lysosomal |
| Regulator | inositol(myo)-1(or 4)-monophosphatase 2 |
| Regulator | inositol hexaphosphate kinase 2 |
| Regulator | phosphoinositide-3-kinase, regulatory subunit, |
| | polypeptide 1 (p85 alpha) |
| Regulator | phosphotidylinositol transfer protein |
| Regulator | inositol 1,4,5-triphosphate 3-kinase B |
| Regulator | protein associated with myc |
| Regulator | v-myc myelocytomatosis viral oncogene (avian) |
| Regulator | hydroxyprostaglandin dehydrogenase 15-(NAD) |
| Regulator | prostaglandin-endoperoxide synthetase 1 |
| Regulator | arachidonate lipoxygenase 3 |
| Regulator | prostaglandin D2 synthase 21 kDa (brains) |
| Regulator | ras-related GTP binding D |
| Regulator | retinoblastoma-associated factor 600 |
| Secreted | lectin, galactoside-binding, soluble 3 |
| Secreted | chemokine-like factor superfamily 6 |
| Secreted | chemokine (C—X—C) motif ligand 12 |
| Secreted | angiopoietin-like 4 |
| Secreted | ephrin-A1 |
| Secreted | apolipoprotein E |
| Secreted | putative secreted protein ZSIG11 |
| Signal transduction | link guanine nucleotide exchange factor ii |
| Signal transduction | SPRY domain-containing SOCS box protein |
| | SSB-3 |
| Trafficking, vesicles | reticulon 3 |
| Trafficking, vesicles | chromosome 12 open reading frame 8 |
| Trafficking, vesicles | vesicle amine transport protein 1 homolog |
| Trafficking, vesicles | adaptor-related protein complex 1, gamma 1 |
| | subunit |
| Transcription | GATA binding protein 3 |
| Transcription | SRY (sex determining region Y)-box 9 |
| Transcription | polymerase (RNA) II (DNA directed) |
| | polypeptide J |
| Transcription factor | catenin, beta interacting protein 1 |
| Transcription factor | nuclear factor I/B |
| Transcription factor | v-kit Hardy-Zukerman 4 feline sarcoma viral |
| | oncogene homolog |
| Transcription factor | Kruppel-like factor 4 (gut) |
| Transcription factor | v-ets erythroblastosis virus E26 oncogene |
| | homolog 2 (avian) |
| Transcription factor | MAX interacting protein 1 |
| Transcription factor | zinc finger protein 36, C3H type-like 2 |
| Transcription factor | forkhead box O3A |
| Transcription factor | v-fos FBJ murine osteosarcoma viral oncogene |
| | homolog |
| Transcription factor | proline-rich nuclear receptor coactivator 2 |
| Transcription factor | OGT(O-Glc-NAc-transferase)-interacting |
| | protein, 106 kDa |
| Transcription factor | myogenic factor 3 |
| Transcription factor | delta sleep inducing peptide, immunoreactor |
| Transcription factor | HMG-box transcription factor 1 |
| Transcription factor | v-maf musculoaponeurotic fibrosarcoma |
| | oncogene homolog (avian) |
| Transcription factor | MAX protein |
| Transcription factor | pre-B-cell leukemia transcription factor |
| | interacting protein 1 |
| Transcription factor | homeodomain-only protein |
| Transcription factor | B-cell CLL/lymphoma 6 (zinc finger protein 51) |
| Transcription factor | B-cell CLL/lymphoma 11A (zinc finger protein) |
| Transcription factor | MYST histone acetyltransferase (monocytic |
| | leukemia) 3 |
| Transcription repressor | cellular repressor of E1A-stimulated genes |
| Transcription repressor | transcription factor 8 (represses interleukin 2 |
| | expression) |
| Translation | membrane protein expressed in epithelial-like |
| | lung adenocarcinoma |
| Translation | ribosomal protein L15 |
| Translation | eukaryotic translation initiation factor 4A, |
| | isoform 2 |
| Translation | eukaryotic translation initiation factor 4B |
| Translation | ribosomal protein L3 |
| Translation | glutaminyl-tRNA synthetase |
| Transporter | solute carrier family 31, member 2 |
| Transporter | aldehyde dehydrogenase 3, family member A2 |
| Transporter | ATPase, class V, type 10B |
| Transporter | hypothetical protein FLJ20296 |
| Transporter | solute carrier family 39, member 6 |
| Transporter | solute carrier family 25, member 6 |
| Transporter | solute carrier family 25, member 11 |
| Transporter | solute carrier family 30, member 1 |
| Transporter | ATPase Ca++ transporting, plasma membrane |
| Transporter | solute carrier family 39, member 2 |
| Transporter | solute carrier family 1, member 4 |
| Transporter | aquaporin 9 |
| Transporter | kelch domain containing 2 |
| Transporter | sodium channel, non-voltage-gated 1, beta |
| | (Liddle syndrome) |
| Transporter | ATPase H+ transporting, lysosomal 50/57 kDa, |
| | V1 subunit H |
| Tumor antigen | silver homolog (mouse) |
| Tumor antigen | hepatocellular carcinoma antigen gene 520 |
| Tumor suppressor | phosphatidic acid phosphatase 2A |
| Tumor suppressor | FGF receptor activating protein 1 |
| Unknown | chromosome 6 open reading frame 48 |
| Unknown | chromosome 7 open reading frame 24 |
| Unknown | alpha-2-glycoprotein 1, zinc |
| Unknown | premature ovarian failure 1B |
| Unknown | KIAA0483 protein |
| Unknown | DKFZP586A0522 protein |
| Unknown | chromosome 14 open reading frame 137 |
| Unknown | KIAA 1536 protein |
| Unknown | cysteine-rich hydrophobic domain 2 |
| Unknown | alpha-2-glycoprotein |
| Unknown | WD repeat domain 26 |
| Unknown | KIAA0930 protein |
| Unknown | SLAc2-B |
| Unknown | HGFL gene |
| Unknown | KIAA0404 protein |
| Unknown | KIAA1815 |
| Unknown | chromosome 6 open reading frame 79 |
| Unknown | Nedd4 binding protein 1 |
| Unknown | KIAA1102 protein |
| Unknown | breakpoint cluster region |
| Unknown/hypothetical | hypothetical protein MGC10940 |
| Unknown/hypothetical | hypothetical protein FLJ22679 |
| Unknown/hypothetical | hypothetical protein MGC11308 |
| Unknown/hypothetical | hypothetical protein FLJ10134 |
| Unknown/hypothetical | hypothetical protein FLJ10901 |
| Unknown/hypothetical | hypothetical protein LOC149603 |
| Unknown/hypothetical | hypothetical protein from clone 643MGC10940 |
| Unknown/hypothetical | hypothetical protein LOC149427 |
| Unknown/hypothetical | hypothetical protein MGC3222 |
| Unknown/hypothetical | hypothetical protein DKFZp43K1210 |
| Unknown/hypothetical | hypothetical protein SP192 |
| Unknown/hypothetical | hypothetical protein FLJ10116 |

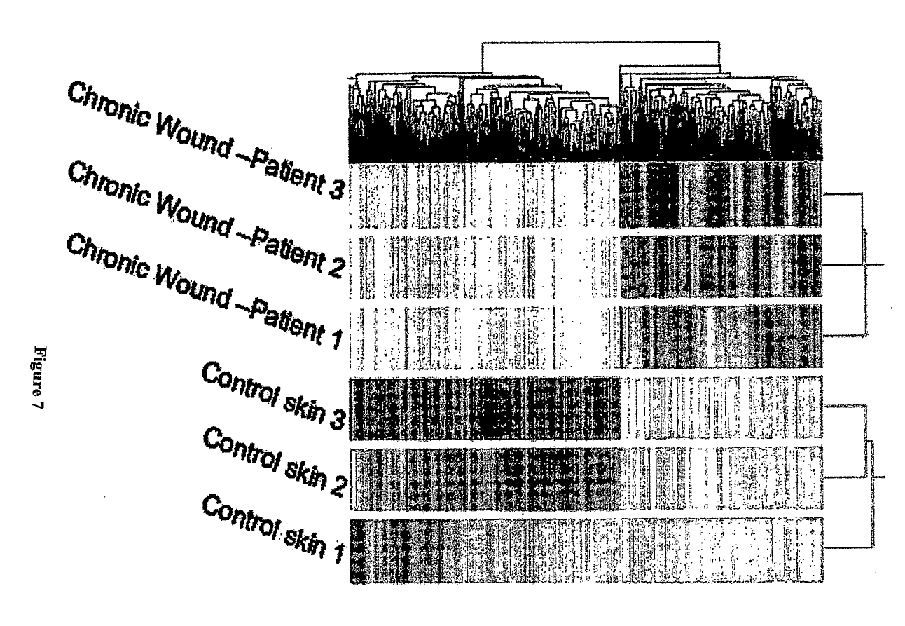

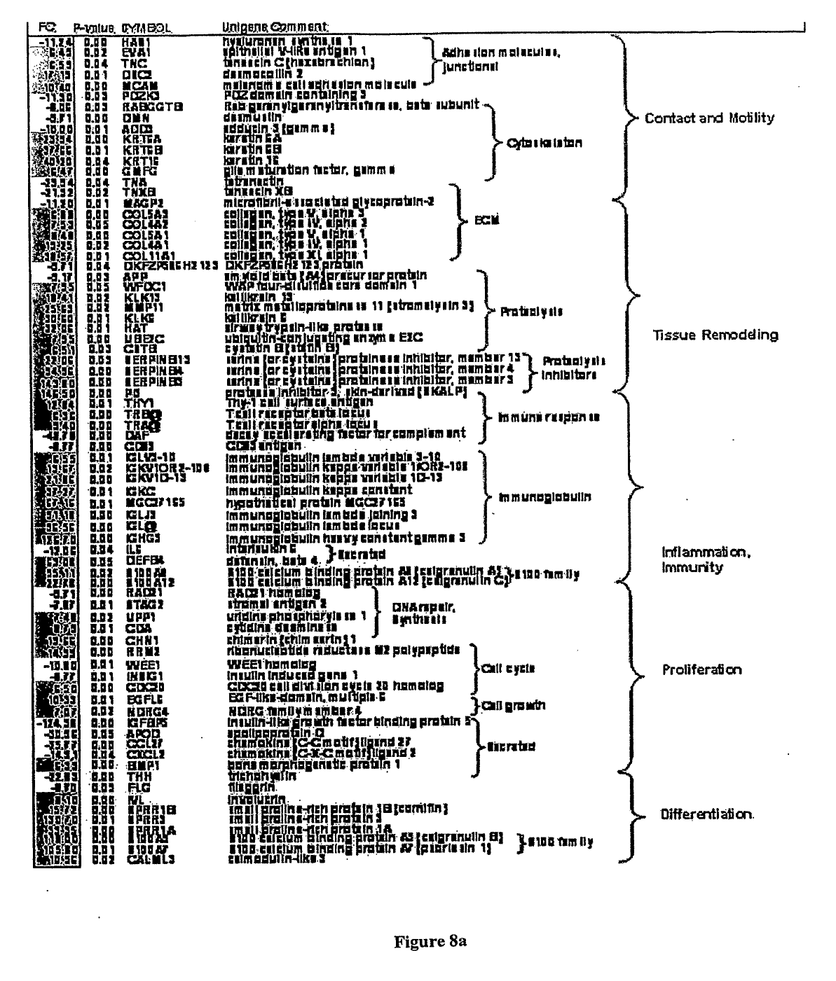

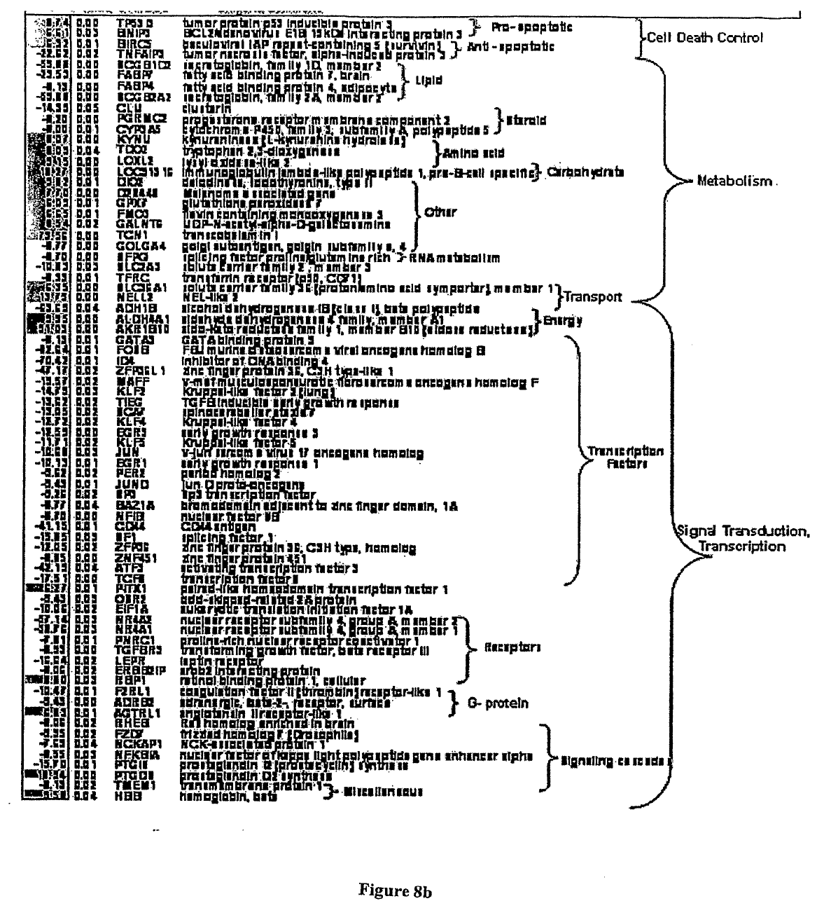

Moreover, there are 100 genes that are the most differentially regulated between the skin of chronic non-healing wounds (NHE) and normal healthy skin. FIG. 8 shows these genes, 50 of which are the most up-regulated in chronic non-healing wound skin as compared to normal skin, and 50 of which are the most down-regulated.

Normal basal epidermal keratinocytes are proliferating and as they exit the basal cell compartment, they commit to a differentiation program. Keratinocyte differentiation requires DNA degradation, nuclear destruction, and substantial proteolytic activity that leads to cell death and the formation of the cornified layer. Most of the 100 differentially regulated genes fall into one of the three main biological processes of keratinocytes: proliferation; differentiation; and apoptosis; thus, showing that these processes are aberrantly regulated in the cells of tissue from chronic non-healing wounds.

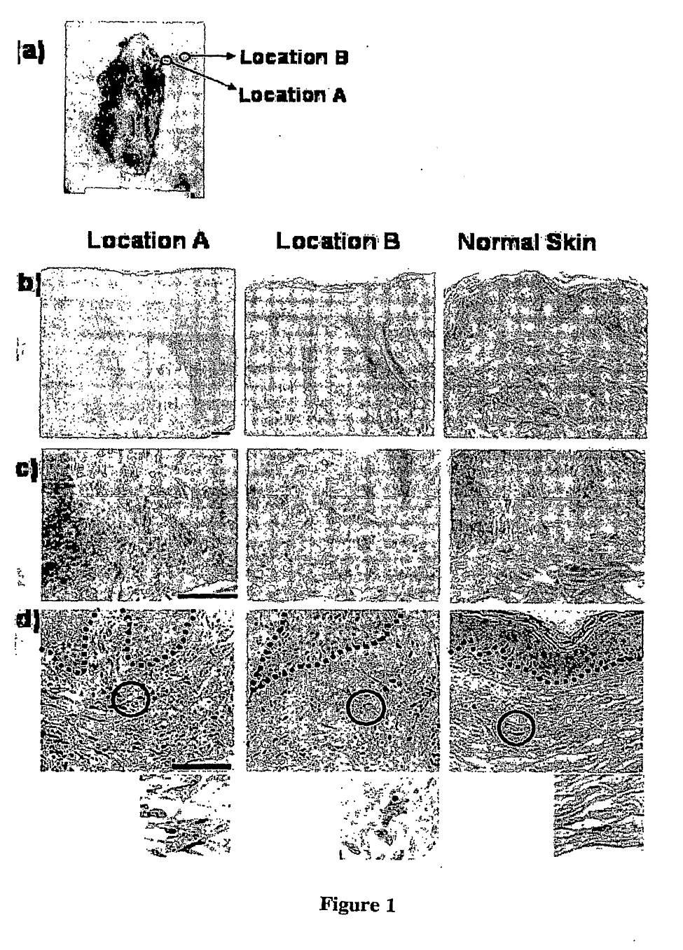

As shown in Examples 1 and 7, chronic wound tissue exhibits a specific morphology. Chronic wound tissue exhibits thick hyperproliferative epidermis with hyperkeratotic (hypertrophy of the cornified layer of skin) and parakeratotic (presence of nuclei in the cornified layer) epidermis (FIG. 1(b)). This morphology indicates aberrant proliferation and improper keratinocyte differentiation (Stojadinovic et al. (2005) Am. J. Pathol. 167:59-69). Results from the microarray analysis confirm that keratinocytes in chronic wound epidermis do not execute either of these processes in a proper manner.

Studies from transgenic mice suggest that differential expression of desmosomal proteins within the epidermis participate in the regulation of the tissue proliferation and differentiation (Brennan et al. (2007) J. Cell Science 120:758-771; Hardman et al. (2005) Mol Cell. Biol. 25:969-978; Smith et al. (2004) Biochem. J. 380:757-765; Merritt et al. (2002) Mol. Cell. Biol. 22:5846-5858; Garrod et al. (1996) Curr. Opin. Cell Biol. 8:670-678). In agreement with these findings, the microarray analysis showed desmosomal molecules are differentially regulated in chronic wounds as compared to normal skin. Specifically, desmosomal cadherin desmocollin 2 (Dsc2) was up-regulated in chronic wounds, while desmocollin 3 (Dsc3) was down-regulated. Desmoglein 3 (Dsg3) was up-regulated and desmoglein 2 (Dsg2) down-regulated. When human Dsg3 was over-expressed under control of keratin1 promoter in suprabasal epidermis of transgenic mice, histological analysis of the skin revealed hyperproliferative epidermis with hyper- and para-keratosis along with abnormal epidermal differentiation (Merritt (2002)). This suggests that in chronic wounds Dsg3 is up-regulated and expressed through the hyperproliferative epidermis, and that the atypical expression of the desmosomal molecules plays a role in epidermal morphogenesis and altered keratinocyte differentiation. Desmoplakin (DP) and plakophilin 2 (PKP2), additional desmosomal molecules, were down-regulated.

Moreover, keratinocyte differentiation markers, keratin 1 (K1) and keratin 10 (K10), were also shown to be down-regulated in chronic non-healing wound tissue by microarray analysis, the down-regulation of the latter protein being confirmed by immunohistochemistry analysis. Additional differentiation markers, filaggrin (FLG) and thrichohyalin (THH) that associate with the keratin cytoskeleton during terminal differentiation were also down-regulated, the down-regulation of the former protein being confirmed by immunohistochemistry analysis.

Involucrin (IVL), a major early cross-linked component of the cornified envelope, and small proline rich proteins (SPRR1A, SPRR1B, SPRR2B, AND SPRR3) were up-regulated, the increased expression of the former protein in chronic wound tissue being confirmed by immunohistochemistry analysis. Transglutaminase 1 (TGM1), one of the enzymes responsible for crosslinking the SPRR proteins and involucrin in to the cornified envelope found in proliferating keratinocytes, but more abundantly expressed in differentiating keratinocytes, was up-regulated. These data suggest improper cornified envelope assembly in chronic wound epidermis (Steinert et al. (1997) J. Biol. Chem. 272:2021-2030).

S100A7, a gene which is part of the human epidermal differentiation complex (EDC) and the S100 family, and S100A8 and S110A9 were also among the 50 most up-regulated genes in the skin of chronic non-healing wounds as found by microarray analysis, the increased expression of the former being confirmed by RT-PCR. These genes are induced in normal primary keratinocytes by high levels of calcium, and found to be highly expressed in inflammatory and hyperproliferative skin diseases (Martinsson et al. (2005) Exp. Dermatol. 14:161-168; Eckert et al. (2004) J. Invest. Dermatol. 123:341-355; Marenholz et al. (2001) Genome Res. 11:341-355).

Kuppel-like factor (KLF4) was down-regulated in the chronic non-healing wound tissue. KLF4 is a transcription factor expressed in the differentiated layers of epidermis important in the establishment of skin barrier function and expression and cross-linking of cornified envelope proteins (Segre et al. (2003) Curr. Opin. Cell Biol. 15:776-782; Bazzoni et al. (2002) J. Cell. Biol. 156:947-949). Manic Fringe protein (MFNG), a protein whose expression is normally restricted to the proliferative basal layer during embryonic epidermal stratification (Thelu et al. (1998) J. Invest. Dermatol. 111:903-906), was up-regulated. This finding, along with the presence of mitoticaly active cells in the suprabasal layer, suggests its role in the induction of keratinocyte proliferation.

NOTCH-2 was downregulated. This protein is involved in the Notch signaling pathway that has been shown to play a role in defining different steps of keratinocyte differentiation (Rangarajan et al. (2001) Embo J. 20:3427-3436; Thelu (1998)).

Phospholipase D (PLD) has been implicated in late keratinocyte differentiation (Jung et al. (1999) Carcinogenesis 20:569-576). PLD1 was found to be down-regulated in chronic wound tissue and PLD2 up-regulated. Moreover, PLD1 mRNA levels are increased during differentiation (Nakashima et al. (1999) Chem. Phys. Lipids 98:153-164), and the highest level of PLD1 expression is found in the more differentiated layers of epidermis (Griner et al. (1999) J. Biol. Chem. 274:4663-460). The finding by microarray analysis of the down-regulation of PLD1 in chronic wound tissue suggests that there are less differentiated keratinocytes in chronic wounds.

Kalikrein 6 (KLK6), implicated in keratinocyte proliferation and differentiation and the pathogenesis of psoriasis (Kishibe et al. (2007) J. Biol. Chem. 282:5834-5841), was found to be up-regulated by microarray analysis, and confirmed by RT-PCR.

Among newly identified potential markers of keratinocyte terminal differentiation (Radoja et al. (2006) Physiol. Genomics 27:65-78), protease inhibitor 3, skin-derived (SKALP, PI3), oxysterol binding protein-like 8 (OSBPL8), adducing 3 (ADD3), early growth response 3 (EGR3), inhibitor of DNA binding 4 (ID4), occluding (OCLN) and decay accelerating factor for complement (DAF) were found to be down-regulated in chronic non-healing wound tissue, whereas septin (SEPT_{—}8), serine/threonine kinase 10 (STK 10), and serine/cysteine proteinase inhibitor, clade B, member 3 (SERPINB3) were up-regulated. These data indicate that aberrant cornified envelope assembly and incomplete terminal differentiation play a role in the pathogenesis of chronic non-healing wounds.

One of the key goals of keratinocyte terminal differentiation is to form a physical barrier that acts as a permeability barrier against water loss, foreign microbes, and toxins. The two important components of the barrier function of the skin is cornified cell envelope and recently introduced tight junctions (TJs) (Bazzoni (2002)). Tight junctions in the skin are complex structures localized in the granular layer and are composed of transmembrane (claudins 1-20, ocludin) and plaque (Symplekin and ZO 1-3) proteins (Denning (2007) J. Invest. Dermatol. 127:742-744; Brandner et al. (2006) Skin Pharmacol. Physiol. 19:71-77). It was found by microarray analysis that many of the different structural proteins of TJ are down-regulated in chronic wound skin as compared to normal skin. This suggests loss of permeability function in the epidermis of chronic wounds. Studies using knock-out mice for different claudins found that while there was TJ formation in the KO mice, the TJ function was completely altered (Furuse et al. (2002) J. Cell. Biol. 156:1099-1111; Pummi et al. (2001) J. Invest. Dermatol 117:1050-1058). These down-regulated TJ proteins include: tight junction protein 3 (TJP3); tight junction proteian, zona ocludens 3 (ZO3); spectrin 1 (SPTBN1); multiple PDZ domain protein (MUPP1); InaD-like protein (INADL); occluding (OCLN); claudin 5 (CLDN5); and claudin 8 (CLDN 8). Only symplekin (SYMPK) was up-regulated.

Formation of TJs in epidermis, as part of differentiation, is a precisely spatiotemporally regulated process. Important components of this regulation include polarity complex Par3, Par6, atypical PKC-iota, and CDC42 (Schneeberger et al. (2004) J. Physiol. Cell. Physiol. 286:C1213-1228). Recent findings suggest that the activity of this complex in the granular layer of the epidermis is necessary for TJs formation and keratinocyte differentation (Helfrich et al. (2007) J. Invest. Dermatol. 127:782-791). Furthermore, during calcium induced differentiation of keratinocytes, atypical PKC-iota was found necessary for the establishment of barrier formation. This complex has characteristic redistribution during wound healing and may also be an endogenous regulator of asymetric cell division of basal keratinoctyes (Denning (2007); Lechler et al. (2005) Nature 437:275-280). Asymmetric skin division promotes stratification and wound healing in the skin by keeping balance between basal proliferation and differentiation. PKC-iota and CDC42 were found to be down-regulated in chronic wound tissue as compared to normal skin, indicating a loss of cell polarity, further indicating a loss of balance between basal proliferation and differentiation, resulting in deregulation of TJ formation.

In mammalian cells, a crucial checkpoint control for proliferation is provided by pocket proteins of the retinoblastoma (Rb) family (Scherr (1996) Science 274:1672-1677; Weinberg (1996) Cell 81:323-330). All three pocket proteins of the Rb family, Rb, p107, and p130 were found to be down-regulated in chronic wound tissue by microarray analysis. Cyclin B1, cyclin D2, cyclin A2, cyclin F, and cyclin M4 were upregulated, as was CDC2, suggesting an increase of CDC2/cyclin B1 and CDC2/cyclin A2 complexes and the promotion of both cell cycle G1/S and G2/M transitions. The microarray data also suggests that there is a loss of cell cycle checkpoint regulation in the epidermis of chronic non-healing wounds. Checkpoint suppressor (CHES1) and WEE1 were down-regulated in chronic wound tissue. WEE1 catalyzes the inhibitory tyrosine phosphorylation of CDC2/cyclinB kinase, and appears to coordinate the transition between DNA replication and mitosis by protecting the nucleus from cytoplasmically activated CDC2 kinase. Without being bound by any theory, the up-regulation of CDC and cyclin B coupled with the loss of inhibitory phosphorylation, may contribute to the hyperproliferative phenotype of chronic wound tissue.

Cyclin D1 was down-regulated in chronic non-healing wound tissue. Over-expression of this gene is frequently observed in a variety of tumors, and may contribute to tumorgenesis. Moreover, EIF4E, which promotes the nuclear export of cyclin D1 is also down-regulated. EIF4E, a translation initiation factor, is a critical modulator of cellular growth, and levels are often elevated in tumors (Culjkovic et al. (2005) J. Cell. Biol. 169:245-256).

Two of the cyclin-dependent kinase inhibitors, CDKNB and CDKN3, were up-regulated. Keratins K6 and K16 were up-regulated, indicating keratinocyte activation.

Among secreted molecules, insulin-like growth factor binding protein (IGFBP5) was among the 50 most down-regulated genes in chronic wounds. Bone morphogenetic proteins (BMP) were differently regulated. BMP2 and BMP7 were down-regulated in chronic wound tissue as shown by both microarray analysis and RT-PCR. In normal human keratinocytes, BMP2 inhibits cell proliferation and promotes terminal differentiation (Gosselet et al. (2007) Cell Signal 19:731-739). The down-regulation of BMP2 in chronic wounds may contribute to the keratinocyte hyperproliferation and have an inhibitory effect on terminal differentiation. The expression of BMP1 was up-regulated.

Leptin enhances wound re-epithelialization (Frank et al. (2000) J. Clin. Invest. 106:510-509). The leptin receptor was found to be down-regulated.

Microarray analysis showed angiogenesis factors, vascular endothelial growth factors (VEGF), epiregulin (EREG) and angiopoetin-like 6 (ANGPTL6) were all down-regulated. ANGPTL6 promotes epidermal proliferation, remodeling, and regeneration (Oike et al. (2003) PNAS 100:9494-9499). Other pro-angiogenic growth factors and receptors were found to be up-regulated in chronic wound tissue such as platelet-derived endothelial cell growth factor (ECGF1), receptor neuropilin (NRP1), and stromal cell-derived factors 1-alpha (CXCL12, SDF-1α). SDF-1α has an important role in homing endothelial progenitor cells.

The microarray analysis showed the strong down-regulation of apolipoprotein D (APOD) (associated with suprabasal differentiated keratinocytes (Radoja (2006)) and the strong up-regulation of defensin B4 (DEFB4) (associated with benign hyperplasia in skin (Haider et al. (2006) J. Invest. Dermatol. 126:869-881) in chronic wound tissue. These data were confirmed by RT-PCR.

Chemokines that mediate T cell chemotaxis were down-regulated, as was the expression of cutaneous T-cell attracting chemokine (CCL27) and IL-7, essential for memory T-cell generation. The expression of the IL-7 receptor was up-regulated, as was the expression of platelet-derived growth factors, PDGFB and PDGFA. The expression of TGFB2, TGFBR3, FGF13, and IL-6 was down-regulated in chronic wound skin. IL deficient mice display significantly delayed cutaneous wound closure (Gallucci et al. (2006) J. Invest. Dermatol. 126:561-568).

The stromelysin-3 gene (MMP-11) was up-regulated as found by microarray analysis and confirmed by RT-PCR. It has been suggested that MMP-11 expression may be under the control of factors produced by inflammatory cells during wound healing and by cancer cells during carcinoma progression (Basset et al. (1993) Breast Cancer Res. Treat. 24:185-193).

Lastly, some of the Fas-mediated apoptosis genes were up-regulated in chronic wound tissue (FASTH, FAF1, PACAP, FASTK) while some were down-regulated (PHLDA2, PCDN6, PTPN13, APAF1). Bcl-2 associated protein, BAX, involved in p53 mediated apoptosis was up-regulated as well as p53 inducible protein 3 (TP53I3). Some inhibitors of apoptosis were down-regulated (BAG4, SERPINB2) while some were up-regulated (NOL3, AVEN, BIRC5). Inhibitor of TNFα mediated apoptosis (TNFAIP3) was down-regulated.

Using the direct correlation between cell biology and gene expression profiles, one can determine a tissue site that is suitable for debriding, i.e., a site with cells which would respond well to debriding. This particular method can be used to determine where in a chronic wound to start debridement as well as to determine the debridement margin. It can also be used to identify tissues with cells that would respond well to other chronic wound treatment. This is an important tool in both further treatment of a chronic wound by pharmaceutical and/or biological agents as well as for testing potential therapeutic agents for chronic wound therapy. If it is known prior to testing such agents that tissues and cells are being targeted that respond well to wound healing stimuli, the outcome of the clinical tests of the agents can be better evaluated. In other words, it would be known that the success or failure of the agent being tested was not related to the cells being targeted and due to some other variable.

To perform this method, one or more tissue samples or biopsies are taken from within or adjacent to a chronic wound. A gene expression profile is then determined for the cells in the site or sites of the tissue biopsies. This gene expression profile is compared to a known gene expression profile from cells that derive from tissue in a site adjacent to the wound (ACW) that is known to respond well to debriding. This known second gene expression profile can be from the non-ulcerated skin adjacent to the wound (ACW) shown in FIG. 2, or from another site adjacent to the wound or away from the wound that has been found to contain cells that respond well to wound healing stimuli. Additional sites can be found by testing the cells in the site for response to wound healing stimuli and determining a gene expression profile from cells with good responses.

Using the correlation between cell biology and gene expression profiles, it can be also determined if a debridement treatment has been successful or if such treatment needs to continue.

If the gene expression profile of a sample tissue biopsy is the same or similar to the cells in the non-healing edge of the wound (NHE), further debridement is required to reach the appropriate cells. If the gene expression profile of the tissue sample is the same or similar to the cells in the non-adjacent non-ulcerative area (ACW), then the debridement was sufficient. Again this information is also useful in both a clinical setting in determining treatment for particular patients, as well as for testing potential therapeutic agents for chronic wound treatment. If it is known prior to testing a therapeutic agent that a wound has been successfully and fully debrided, the outcome of the testing can be better evaluated.

In performing this method, one or more biopsies or tissue samples from in or adjacent to the chronic wound may be taken. It is preferable, but not necessary, that the sample be from any area of the chronic wound where debridement has already been performed. A gene expression profile is then determined for the cells in the site or sites of the tissue biopsies. Once the gene expression profiles for the biopsied tissue are determined, they can be compared to the known gene expression profile of the cells from the adjacent non-ulcerated skin (ACW) found in FIG. 2. However, comparison can also be made to the gene expression profiles of tissue adjacent to the chronic wound that have been shown to have cells with a healthy morphology and/or a good response to wound healing stimuli, or other healthy skin. If the gene expression profile of the biopsied tissue is the same or similar to the gene expression profile of the tissue containing cells with healthy morphology and/or good response to wound healing stimuli, then the debriding has been sufficient and can be terminated.

Any methods known in the art can be used to test for the various biological characteristics of the cells. A preferred method for testing the response to wound healing stimuli is an in vitro wound scratch assay performed on fibroblasts grown from the tissue samples. This method requires growing fibroblasts from the biopsied tissue and once the culture is established, scratching the cells with a sterile pipet or other instrument. The capacity of the cells to respond to the wound healing stimuli is measured by the distance the cells migrate to cover the initial scratch. The further the cells migrate, the better their response to the scratch, i.e., wound healing stimuli. Cells with further migration would be predicted to grow better and heal after surgical debridement.

The preferred method for determining the morphology of the cells is staining by hematoxylin, eosin and/or an antibody such as one for pro-collagen.

The current preferred technology that would be used to determine the gene expression profiles or “bar codes” of the tissue is microarrays. Processing the tissue samples from obtaining a biopsy to obtaining a gene expression “bar code” takes approximately three days. However, under current treatment protocols, this information is still clinically useful as there is often waiting periods in debridement procedures.

The terms “array” or “microarray” are used interchangeably and refer generally to any ordered arrangement (e.g., on a surface or substrate) of different molecules, referred to herein as “probes.” Each different probe of any array is capable of specifically recognizing and/or binding to a particular molecule, which is referred to herein as its “target” in the context of arrays. Examples of typical target molecules that can be detected using microarrays include mRNA transcripts, cRNA molecules, and proteins.

Microarrays are useful for simultaneously detecting the presence, absence and quantity of a plurality of different target molecules in a sample (such as an mRNA preparation isolated from a relevant cell, tissue or organism, or a corresponding cDNA or cRNA preparation). The presence and quantity, or absence, of a probe's target molecule in a sample may be readily determined by analyzing whether (and how much of) a target has bound to a probe at a particular location on the surface or substrate.

In a preferred embodiment, arrays used in the present invention are “addressable arrays” where each different probe is associated with a particular “address.”

The arrays utilized in the present invention are preferably nucleic acid arrays that comprise a plurality of nucleic acid probes immobilized on a surface or substrate. The different nucleic acid probes are complementary to, and therefore can hybridize to, different target nucleic acid molecules in a sample. Thus, such probes can be used to simultaneously detect the presence and quantity of a plurality of different nucleic acid molecules in a sample, to determine the expression of a plurality of different genes, e.g., the presence and abundance of different mRNA molecules, or of nucleic acid molecules derived therefrom (for example, cDNA or cRNA).

There are two major types of microarray technology: spotted cDNA arrays and manufactured oligonucleotide arrays. The Example section below describes the use of a high density oligonucleotide Affymetrix GeneChip® human genome array.

The arrays are preferably reproducible, allowing multiple copies of a given array to be produced and the results from each easily compared to one another. Preferably microarrays are small, usually smaller than 5 cm², and are made from materials that are stable under binding (e.g., nucleic acid hybridization) conditions. A given binding site or unique set of binding sites in the microarray will specifically bind the target (e.g., the mRNA of a single gene in the cell). Although there may be more than one physical binding site (hereinafter “site”) per specific target, for the sake of clarity the discussion below will assume that there is a single site. It will be appreciated that when cDNA complementary to the RNA of a cell is made and hybridized to a microarray under suitable hybridization conditions, the level or degree of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding a nucleic acid product of the gene) that is not transcribed in the cell will have little or no signal, while gene for which the encoded mRNA is highly prevalent will have a relatively strong signal.

By way of example, GeneChip® expression analysis (Affymetrix, Santa Clara, Calif.) generates data for the assessment of gene expression profiles and other biological assays. Oligonucleotide expression arrays simultaneously and quantitatively “interrogate” thousands of mRNA transcripts (genes or ESTs), simplifying large genomic studies. Each transcript can be represented on a probe array by multiple probe pairs to differentiate among closely related members of gene families. Each probe set contains millions of copies of a specific oligonucleotide probe, permitting the accurate and sensitive detection of even low-intensity mRNA hybridization patterns. After hybridization intensity data is captured, e.g., using optical detection systems (e.g., a scanner), software can be used to automatically calculate intensity values for each probe cell. Probe cell intensities can be used to calculate an average intensity for each gene, which correlates with mRNA abundance levels. Expression data can be quickly sorted based on any analysis parameter and displayed in a variety of graphical formats for any selected subset of genes. Gene expression detection technologies include, among others, the research products manufactured and sold by Hewlett-Packard, Perkin-Elmer and Gene Logic.

It is contemplated that technological developments will allow more rapid processing of the RNA from tissue to chips, such as a desktop machine that has been recently reported that allows doctors to access a patient's DNA from a drop of blood in just an hour (Cyranoski (2005) Nature 437:796).

As shown, certain genes are up-regulated or induced in the cells from tissue from chronic non-healing wounds as compared to healthy skin, and certain genes are down-regulated or suppressed. This differential regulation of certain genes can also be used to identify a suitable site for debridement as well as determine if the debridement needs to be continued on a wound.

To perform a method for identifying a suitable site for debridement, one of more tissue samples are taken from within or adjacent to a chronic wound. The expression of a gene or genes known to be differentially regulated in chronic non-healing wound tissue (NHE) as compared to normal skin is determined. If the expression of the gene or genes is identical or similar in the sample, i.e., up-regulated or down-regulated, to the known expression of the gene or genes in the cells from the tissue of chronic non-healing wounds (NHE), then the site is suitable for debridement. If the expression of the gene or genes is identical or similar in the sample, i.e., up-regulated or down-regulated, to the known expression of the gene or genes in the cells of healthy skin, then the site is not suitable for debridement.

To perform a method for determining if a debridement treatment has been successful or if such treatment needs to continue, one of more tissue samples are taken from within or adjacent to a chronic wound. It is preferable, but not necessary, that the sample be from an area of the wound where debridement has already been performed. The expression of a gene or genes known to be differentially regulated in chronic non-healing wound tissue (NHE) as compared to normal skin is determined. If the expression of the gene or genes is identical or similar in the sample, i.e., up-regulated or down-regulated, to the known expression of the gene or genes in the cells from tissue of chronic non-healing wounds (NHE), then further debridement is necessary. If the expression of the gene or genes is identical or similar in the sample, i.e., up-regulated or down-regulated, to the known expression of the gene or genes in the cells of healthy skin, then the debridement has been successful.

Any method known in the art can be used to determine the expression of the gene or genes in the sample. Such methods include, but are not limited to, microarray analysis, RT-PCR, quantitative RT-PCR, immunohistochemistry, Southern, Northern and Western blots.

Genes that are known to be up-regulated or induced in the cells of tissue from chronic non-healing wounds (NHE) as compared to the cells in normal healthy skin, include, but are not limited to desmocollin 2 (Dsc2), desmoglein 3 (Dsg3), involucrin (IVL), small proline rich protein 1A (SPRR1A), small proline rich protein 1B (SPRR1B), small proline rich protein 2B (SPRR2B), small proline rich protein 3 (SPRR3), transglutaminase 1 (TGM1), S100 calcium binding protein A7 (S100A7), S100 calcium binding protein A8 (S100A8), S100 calcium binding protein A9 (S100A9), manic fringe protein (MFNG), phospholipase D 2 (PLD2), kalikrein 6, (KLK6), septin (SEPT_{—}8), serine/threonine kinase 10 (STK10), serine/cysteine proteinase inhibitor, clade B, member 3 (SERPINB3), symplekin (SYMPK), cyclin B1, cyclin D2, cyclin A2, cyclin F, cyclin M4, cell division cycle 2 homolog (CDC2), cyclin dependent kinase inhibitor NB (CDKNB), cyclin dependent kinase inhibitor N3 (CDKN3), keratin 6 (K6), keratin 16 (K16), bone morphogenetic protein 1 (BMP-1), platelet derived endothelial growth factor (ECGF1), receptor neuropilin (NRP1), stromal cell derived factor 1-alpha (SDF-1α), defensin B4 (DEFB4), IL-7 receptor (IL-7R), platelet derived growth factor B (PDGFB), platelet derived growth factor A (PDGFA), Fas-activated serine/theorine kinase (FASTK), Fas (TNFRSF6) associated factor (FAF1), proapoptotic caspase adaptor protein (PCAP), bcl-2 associated protein (BAX), p53 inducible protein (TP5313), nucleolar protein 3(NOL3), apoptosis, caspase activation inhibitor (AVEN), and baculoviral IAP repeat-containing 5 (surivin) (BIRC5).

Genes that are known to be down-regulated or suppressed in the cells of tissue from chronic non-healing wounds (NHE) as compared to cells in normal, healthy skin, include, but are not limited to desmocollin 3 (Dsc3), desmoglein 2 (Dsg2), desmoplakin (DP), plakophilin 2 (PKP2), filaggrin (FLG), thrichohyalin (THH), kuppel-like factor (KLF4), NOTCH, drosophila, homolog OF, 2 (NOTH2), phospholipase D 1 (PLD1), protease inhibitor 3, skin-derived (SKALP, PI3), oxysterol binding protein-like 8 (OSBPL8), adducing 3 (ADD3), early growth response 3 (EGR3), inhibitor of DNA binding 4 (ID4), occluding (OCLN), decay accelerating factor for complement (DAF), tight junction protein, zona ocludens 3 (ZO3), tight junction protein 3 (TJP3), spectrin 1 (SPTBN1), multiple PDZ domain protein (MUPP1), InaD-like protein (INADL), claudin 5 (CLDN5), claudin 8 (CLDN8), protein kinase C-iota (PKC-iota), cell division cycle homolog 42 (CDC42), retinoblastoma protein (Rb), retinoblastoma protein (p107), retinoblastoma protein (p103), checkpoint suppressor (CHES1), WEE1 homolog (WEE1), translation initiation factor (EIF4E), insulin-like growth factor binding protein (IGFBP5), bone morphogenetic protein 2 (BMP2), bone morphogenetic protein 7 (BMP7), leptin receptor (LEPR), vascular endothelial growth factor (VEGF), epiregulin (EREG), angiopoetin-like 6 (ANGPTL6), apolipoprotein D (APOD), cutaneous T cell attracting chemokine 27 (CCL27), IL-7, transforming growth factor, beta 2 (TGFB2), transforming growth factor, beta 3, (TGFBR3), fibroblast growth factor 13 (FGF13), interleukin 6 (IL-6), pleckstrin homology-like domain, family A, member 2 (PHLDA2), programmed cell death (PDCD6), protein tyrosine phosphatase, non-receptor type 13 (APO-1/CD95 (Fas)-associated phophatase (PTPN13), apoptotic peptidase activating factor 1 (APAF1), and TNFα mediated apoptosis inhibitor (TNFAIP3).

### EXAMPLES

### Example 1

### Skin Specimens and Histology

### Materials and Methods

A total of eight skin sample biopsies were obtained from three consented patients with venous reflux ulcers as discarded tissue after debridement procedures. The biopsies were obtained in a blinded fashion, i.e., the wound location was under code. As shown in FIG. 1A, the biopsies were obtained from two distinct locations in the wounds: the non-healing edge (NHE) (location A) and the adjacent non-ulcerated skin (ACW) (location B).

A small portion of the specimens were fixed in formalin and processed for paraffin embedding. The paraffin embedded tissues were sectioned and 5 μm thick sections were stained with hematoxylin and eosin. The sections were also stained with pro-collagen type I antibody M-38 (Developmental Studies Hybridoma Bank at University of Iowa, described in McDonald et al. (1986) J. Clin. Invest. 78:1237-1244) following the published protocol of Stojadinovic et al. (2005) Am. J. Pathol. 167:59-69. The sections were analyzed using a Carl Zeiss microscope (Carl Ziess, Thornwood, N.Y.) and digital images collected using an Adobe TWAIN_{—}32 program.

### Results

The results of the histological staining showed that the locations of non-healing wounds differed in their morphology. FIG. 1(b) shows the results of stained tissue from the epidermis layer. The hematoxylin and eosin stained biopsy obtained from the non-healing edge (NHE) (location A as shown in FIG. 1(a)) showed thick, hyperproliferative epidermis with hyperkeratotic (hypertrophy of the cornified layer of the skin) and parakeratotic (presence of nuclei in the cornified layer) epidermis (FIG. 1(b)). Following the debridement margin towards healthy skin, the morphology of the skin biopsies transformed. Epidermis from adjacent, non-ulcerated skin (ACW) (location B as shown in FIG. 1(a)) was normalized and exhibited a well-defined cornified layer and significantly less hyperproliferation as compared to the non-healing edge. However, it was still more hyperproliferative than epidermis of normal skin that is not part of the wound (FIG. 1(b)).

FIG. 1(c) shows stained tissue from the dermis layer. Epidermal ridges (projections of the epidermis into the dermis) were also present in the adjacent, non-ulcerated skin, although they extended deeper in the dermis than in normal skin. Evidence of fibrosis was also found in both the dermis in the non-healing edge and the non-ulcerated skin adjacent to the wound, although to a lesser extent in the non-ulcerated skin. The dermis of the skin from the non-healing edge exhibited increased cellularity when compared to adjacent, non-ulcerated or normal skin (FIG. 1(c)).

Finally, intracellular pro-collagen was most pronounced in the dermis from the non-healing edge when compared with skin from the adjacent, non-ulcerated area or normal skin (FIG. 1(d)).

In summary, the stained biopsies from non-healing edge of the wound (NHE) (location A) exhibited severe pathogenesis as compared to the adjacent, non-ulcerated skin (ACW) (location B). It was concluded that the biology of the skin within the wound edge differs from healthy skin.

### Example 2

### Total RNA Isolation and Microarray Analysis

### Materials and Methods

Samples from Example 1 were stored in an RNAlater (Ambion) for subsequent RNA isolation. Total RNA from the samples of Example 1 was then isolated using RNeasy (QIAGEN, Valencia, Calif.) following the commercial protocol. Northern Blot analysis was performed to assess the quality of the isolated mRNA. Using RNeasy protocol, 5 μg of total RNA was reversed-transcribed, amplified and labeled. Labeled cRNA was hybridized to GeneChip®) Human Genome U133 arrays (Affymetrix, Santa Clara, Calif.) following commercial protocol. The arrays were washed and stained with anti-biotin streptavidin-phycoerythin labeled antibody using Affymetrix fluidics station and then scanned using the Agilent GeneArray Scanner system (Hewlett-Packard, Palo Alto, Calif.).

Microarray Suite 5.0 (Affymetix) was used for data extraction. Data Mining Tool 3.0 (Affymetrix) was used for further analysis. GeneSpring™ software 5.1 (Silicon Genetics, Santa Clara, Calif.) was used for normalization, fold change calculations, and clustering.

Differential expressions of transcripts were determined by calculating the fold change. Genes were considered regulated if the expression levels differed by more than 2-fold to healing edges. Clustering was performed based upon similarity of the expression pattern in all samples using GeneSpring™.

### Results

Using the Affymetrix HU133 chips and Gene Spring™ software as described above, hybridizations of the eight samples were performed, four from the non-healing edges (NHE) (location A) and four from the non-ulcerated skin (ACW) (location B). The various samples were compared and a specific transcriptional, i.e., gene expression, profile was obtained. Gene expression was visualized by generating gene trees, a graphic representation in which sample are grouped based on the similarity of their gene expression profiles. The dark gray lines represent up-regulated genes, the lighter gray lines represent down-regulated genes and the lightest gray lines represent expressed genes, but ones that are not significantly regulated. This method allows the overall visualization of the entire gene expression pattern, rather than specific gene regulation. Using this method, it is shown that the expression patterns from the samples from the non-healing edge (location A) are similar, while the expression patterns from the samples taken from the adjacent, non-ulcerated skin (location B) are similar to each other but quite different than the pattern from the samples from the non-healing edge (FIG. 2).

These gene expression pattern profiles coupled with the tissue morphology studies in Example 1 show that the cells from the two different wound locations exhibit different biological features.

### Example 3

### Primary Fibroblast Cell Culture

### Materials and Methods

The 5 mm biopsies obtained from three patients during debridement procedure were used to establish fibroblast cultures. The biopsies were obtained from two different locations: non-healing wound edge (NHE) and adjacent non-ulcerated skin (ACW). The underlying fat beneath the skin was removed, and the tissue washed six times in phosphate buffered saline (PBS), and minced into pieces approximately 1 mm² in size. The tissue pieces were placed in 75 cm² tissue culture flasks containing Dulbecco's modified Eagle medium (DMEM) supplemented with 10% serum, and a penecillin/streptamycin/gentamycin mixture. After several days in culture, fibroblasts were observed sprouting from the tissue explants. The mono layer was trypsinized to separate the tissue explants from the cells. Dermal fibroblasts were then seeded in DMEM with 10% serum and the penecillin/streptamycin/gentamycin mixture. The fibroblasts were propagated by trypsinization until the fourth passage.

### Results

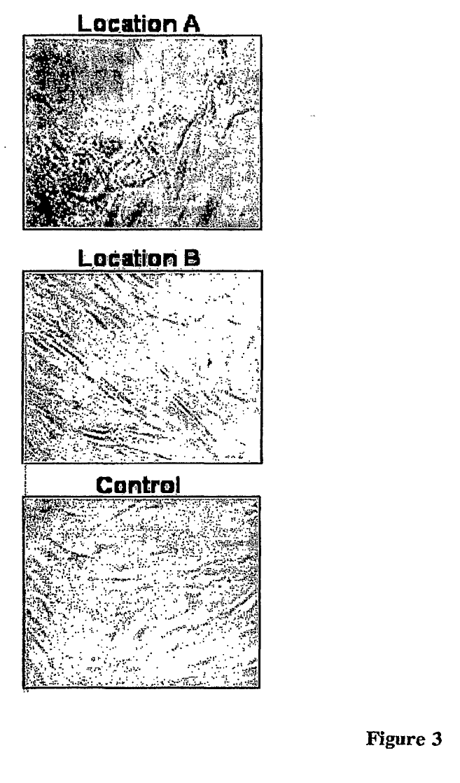

The fibroblasts grown from the tissue at the non-healing edge of the chronic wounds (NHE) exhibited pathogenic phenotypes, whereas the fibroblasts grown from the adjacent non-ulcerated area (ACW) (location B) had a phenotype similar to primary fibroblasts obtained from healthy skin (control) (FIG. 3). The fibroblasts from the non-healing edge of the chronic wound were misshaped, inflated with large nuclei, and clumped together as compared to normal cells (FIG. 3).

### Example 4

### Wound Scratch Assay

### Materials and Methods

The primary human dermal fibroblasts described in Example 3 were grown to 80% confluency. Cells were transferred to basal medium containing DMEM with 5% stripped serum (Radoja et al. (2000) Mol. Cell. Biol. 20:4328-4339) 24 hours prior to the experiment. On day 0, the cells were treated with 8 μg/ml of Mitomycin C (ICN) for one hour and washed with 1×PBS prior to scratch.

Scratches were performed using sterile yellow pipet tips and photographed using a Carl Zeiss microscope and a Sony digital camera. Cells were further incubated for 4, 8 and 24 hours and re-photographed in the same fields as initially done on day 0. Cell migration was quantified using a Sigma Scan Program. Measurements were taken for each experimental condition and expressed as a percentage of distance covered by the cells moving into the scratch wound area for each time point after wounding. Three images are analyzed per condition and time point, and averages and standard deviations were calculated.

### Results

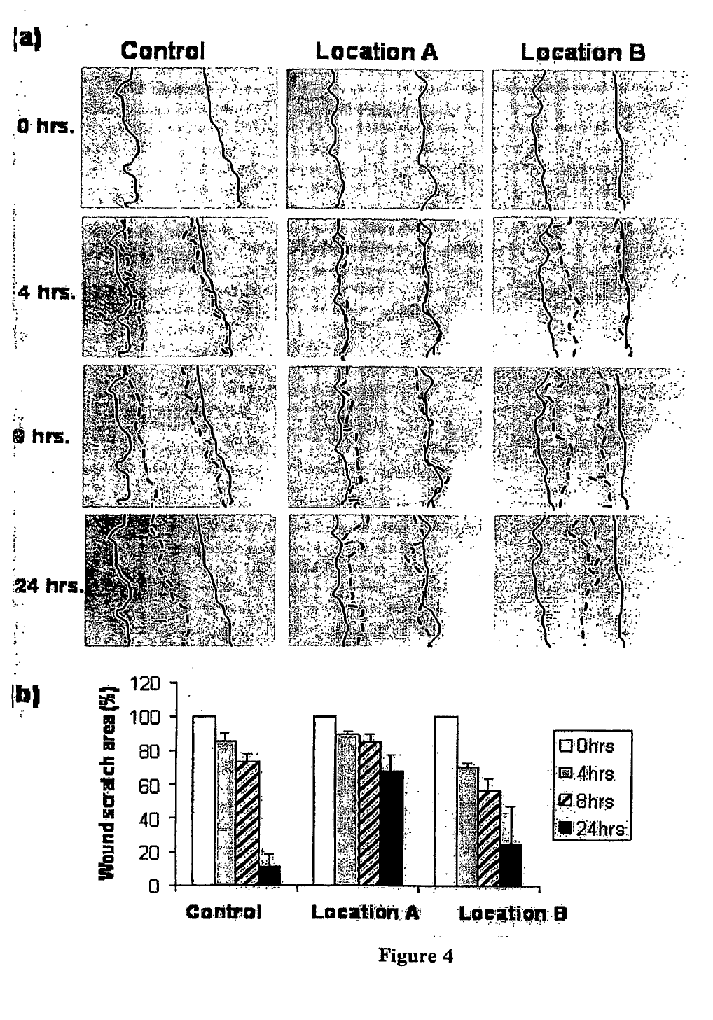

The fibroblasts grown from the non-healing edge tissue (NHE) (location A) have the slowest migration rate, covering only 33% of the initial scratch in 24 hours. Fibroblasts grown from the adjacent, non-ulcerated tissue (ACW) (location B) covered 75%, only slightly less than the control which closed 89% of the scratched area (FIG. 4).

The results from Examples 1-4 indicate a direct correlation between specific location within the wound, cellular biology, cellular response to wounding, and gene expression profile.

### Example 5

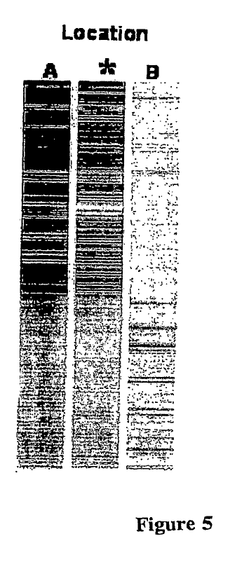

Using the microarray analysis described in Example 2, gene expression patterns were obtained for samples from the non-healing edge (NHE) (location A), the adjacent non-ulcerated tissue (ACW) (location B), and an additional sample from an intermediate location between locations A and B (location *). The gene expression patterns for each sample are found in FIG. 5. As can be seen from the Figure, the gene expression pattern of the intermediate sample (indicated by an “*”) was more similar to the gene expression pattern of non-healing edge sample, indicating that debridement procedure needed to proceed further, until a healing pattern, similar to that of location B, is detected. This data suggest that gene expression pattern changes may serve as an indication of the pathogenic progress within the wound, which can further guide the extent of the debridement.

### Example 6

### Further Analysis of Expression of Specific Genes

### Materials and Methods

Further analysis of the actual genes being up-regulated and down-regulated in the gene expression profiles obtained in Example 2 were done using Microarray Suite 5.0 (Affymetix) for data extraction, Data Mining Tool 3.0 (Affymetrix) for further analysis and GeneSpring™ software 5.1 (Silicon Genetics) for normalization, fold change calculations, and clustering.

Differential expressions of transcripts were determined by calculating the fold change. To compare data from multiple arrays, the signal of each probe array was scaled to the same target intensity value. Genes were considered regulated if the expression levels differed by more than 2-fold to healing edges at any time point. Fold changes obtained from the first and second experiments were averaged and determined regulated if the fold changes were more than 2 or less than 2. Clustering was performed based upon similarity of the expression pattern in all samples using GeneSpring™.

An extensive gene annotation table was produced describing the molecular function and biological category of the genes present on the Affymetrix Human Genome chip based upon data from J. M. Ruillard and the Gene Ontology Consortium Data available on the World Wide Web at cgap.nci.nih.gov/Genes/GOBrowser and ot.ped.med.umich.edu:2000/ourimage/pub/shared/JMR_pub_affyannot.html.

### Results

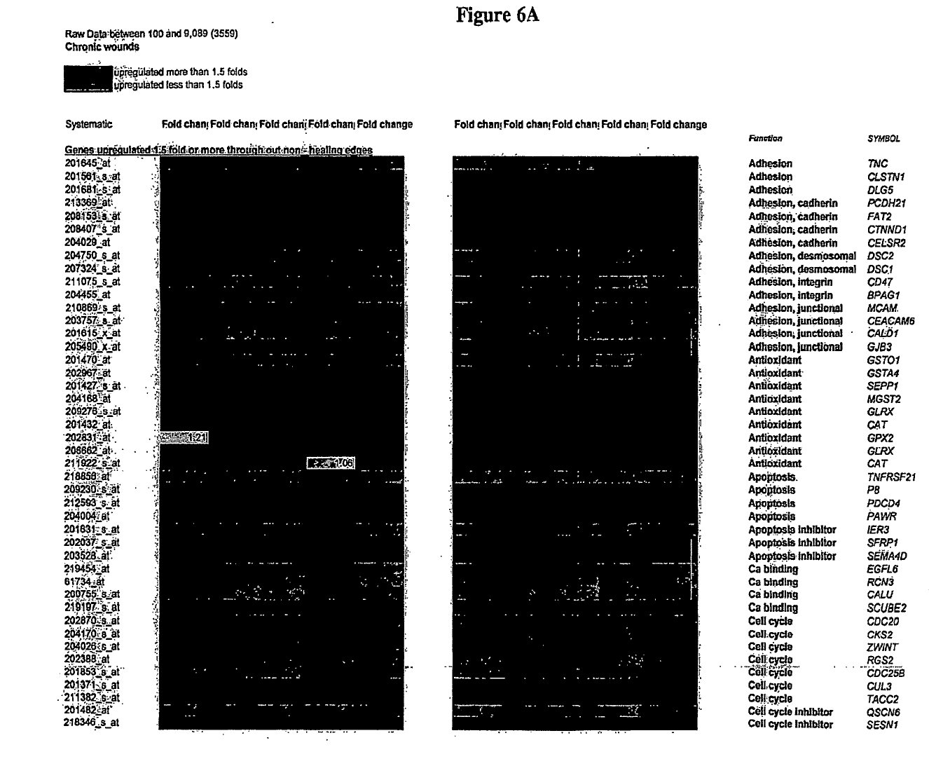

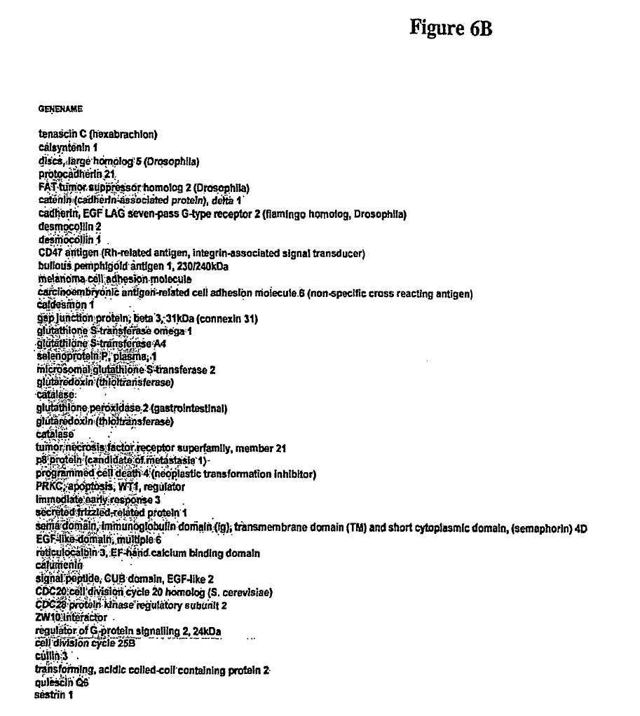

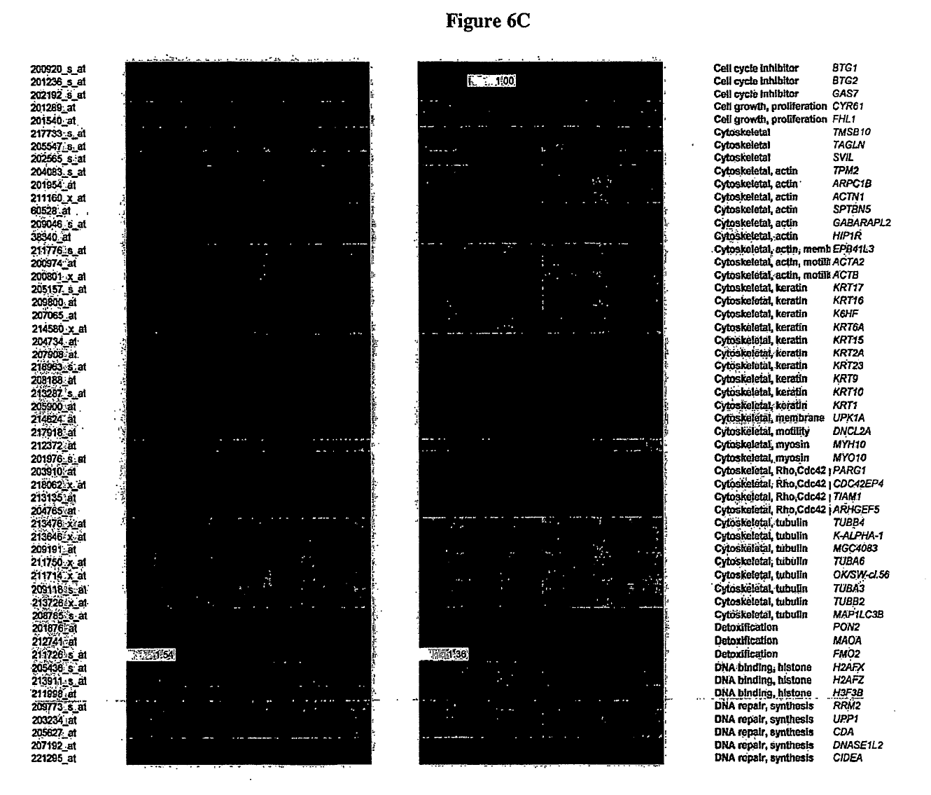

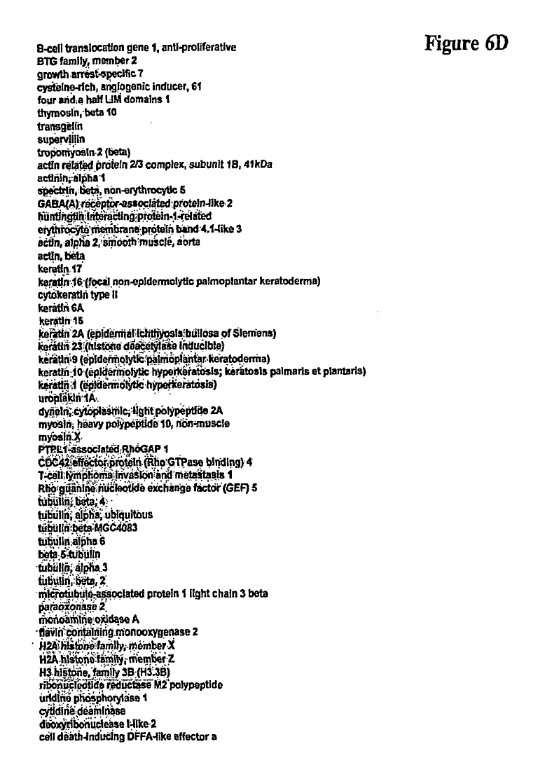

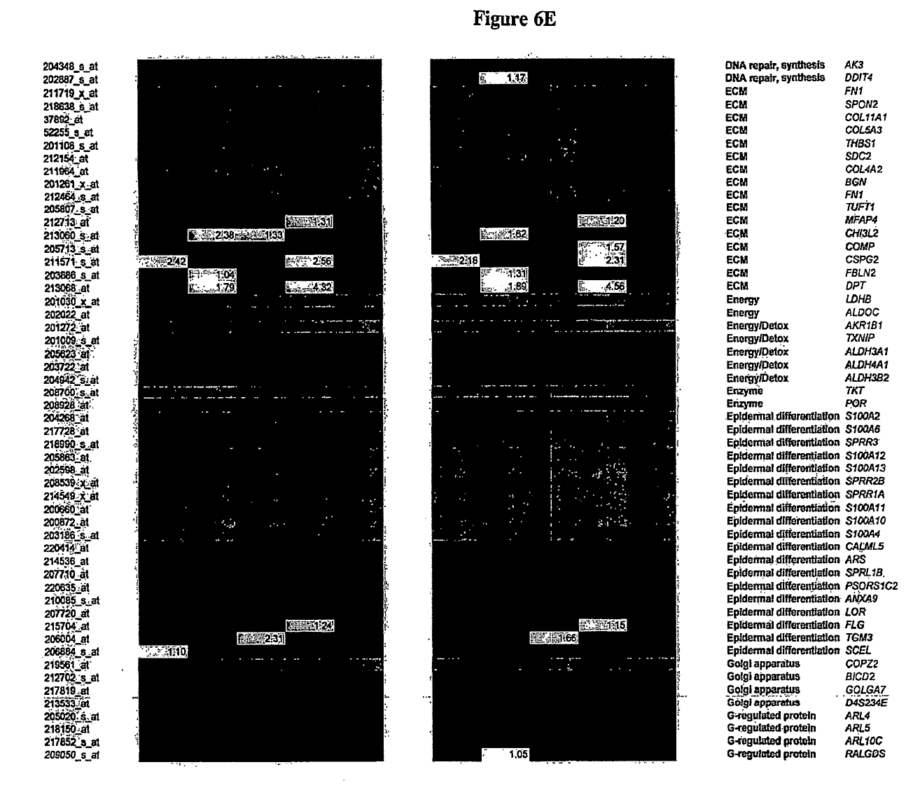

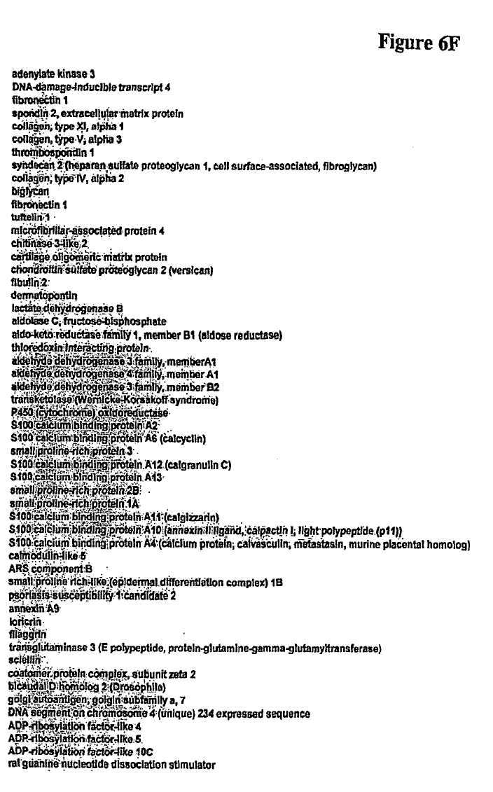

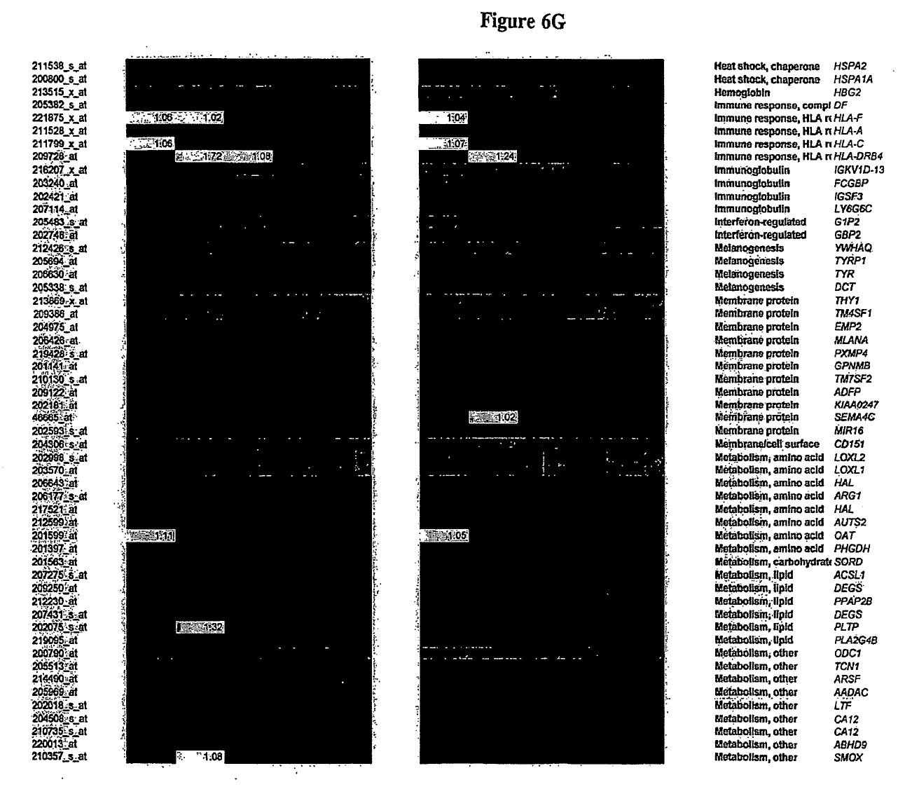

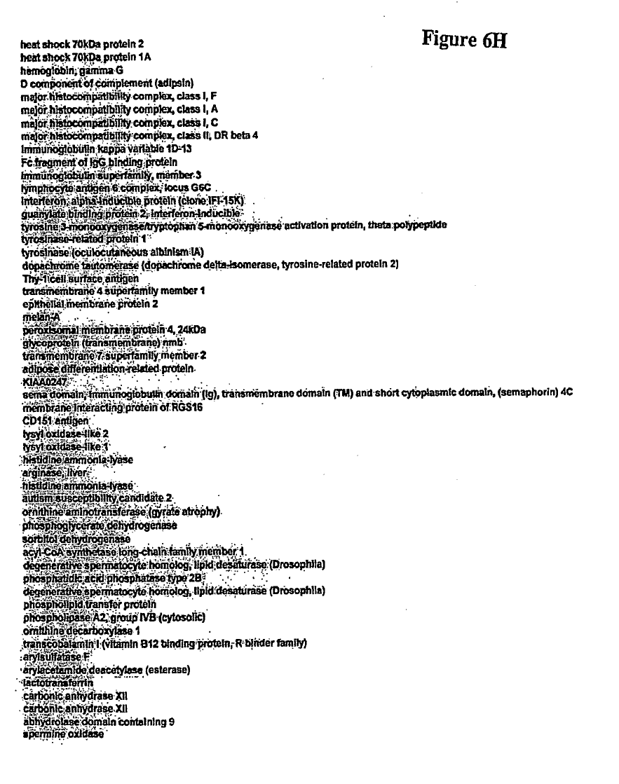

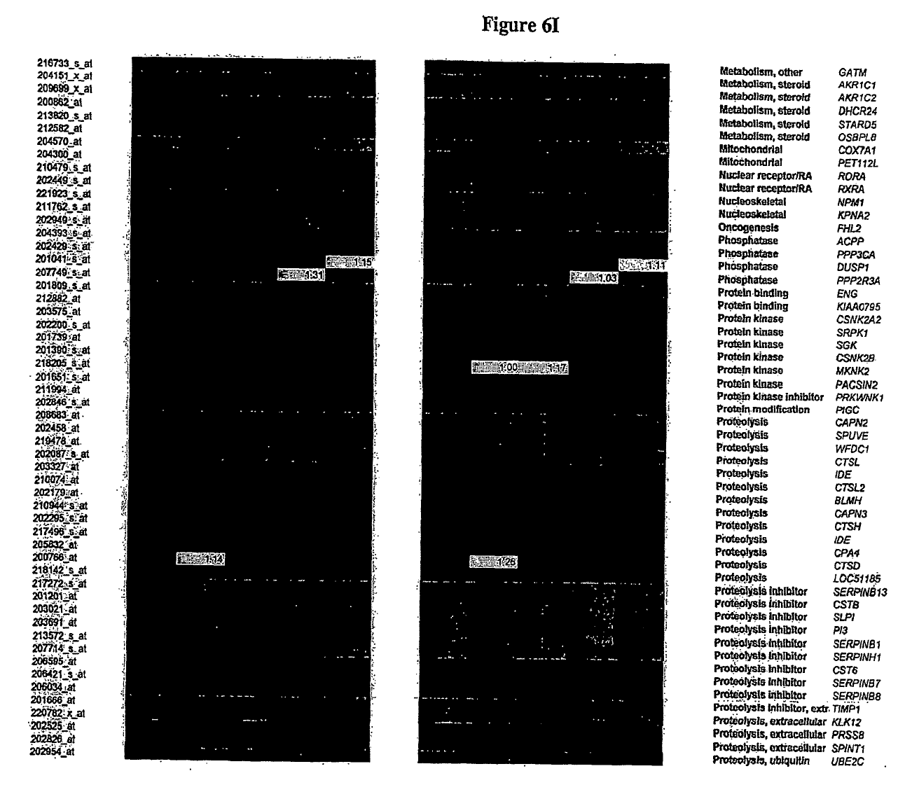

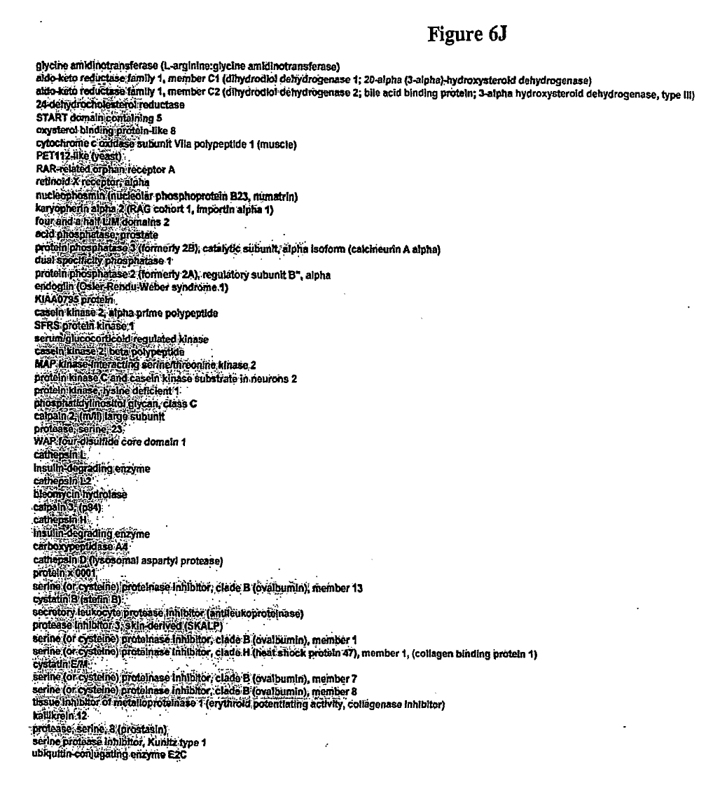

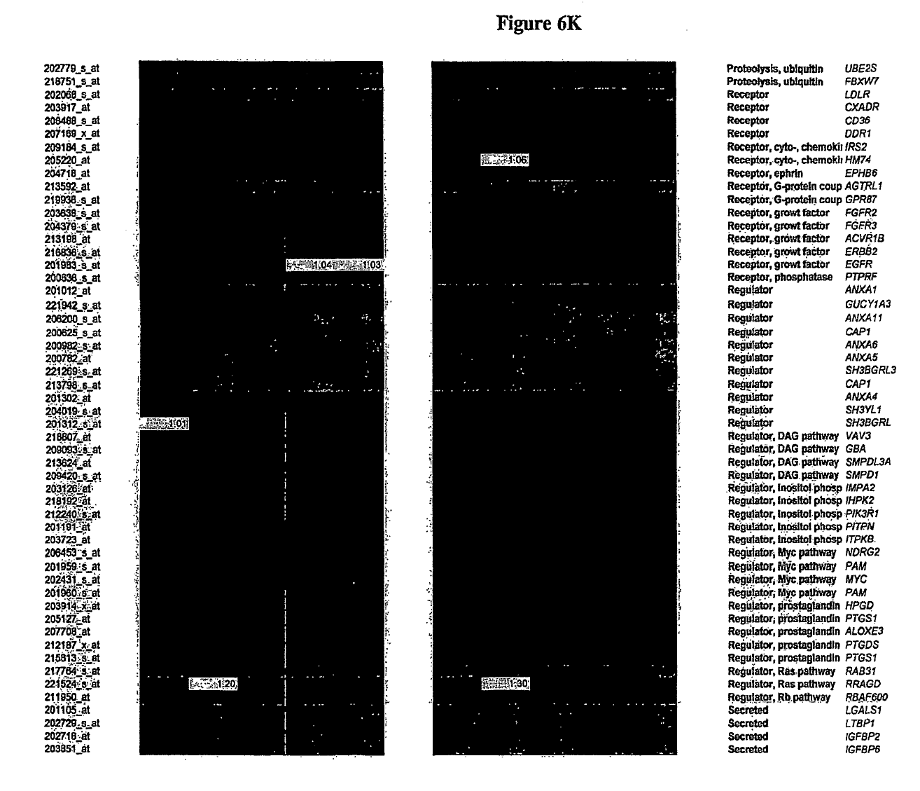

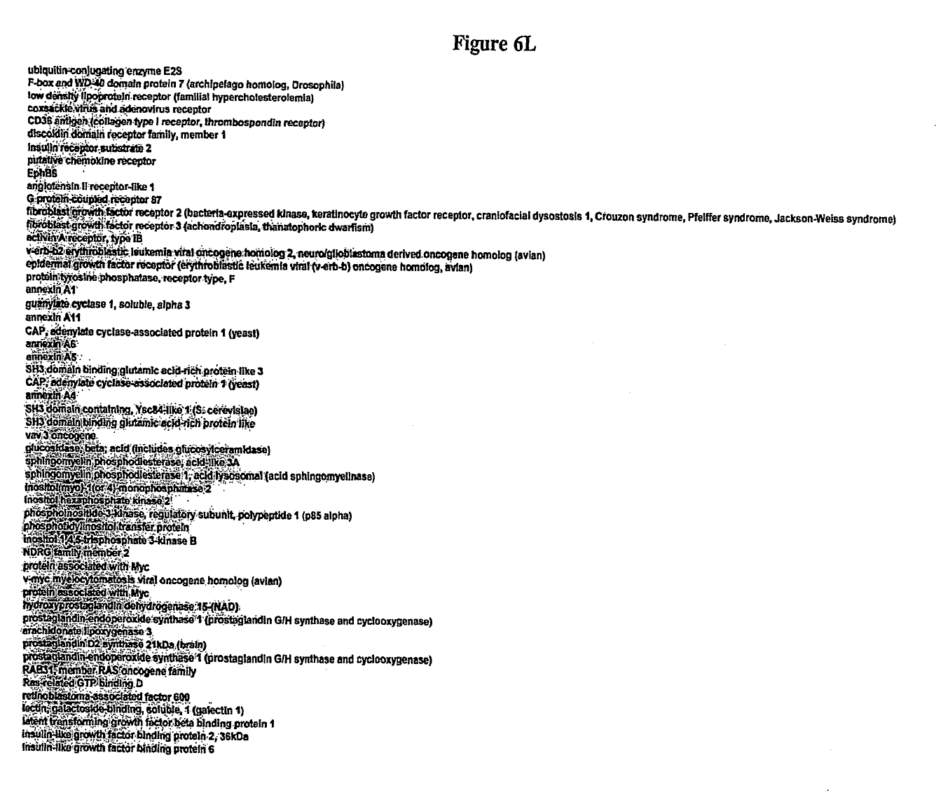

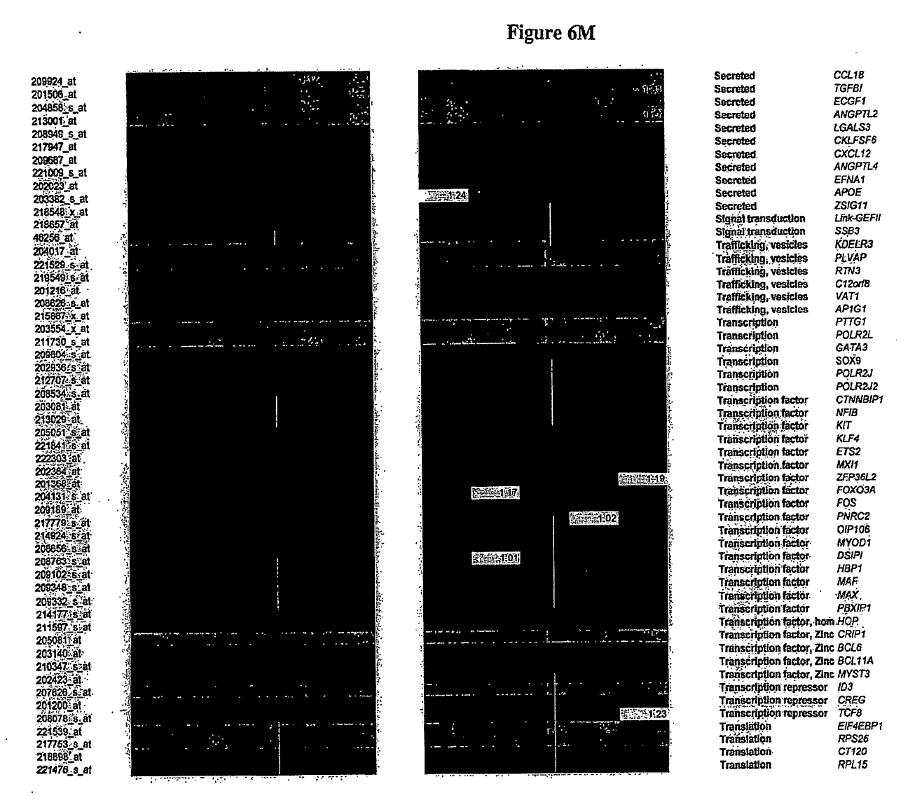

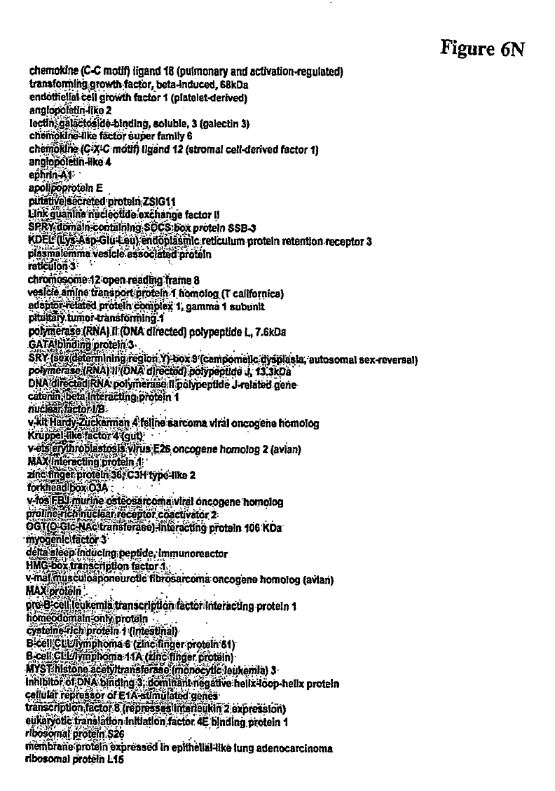

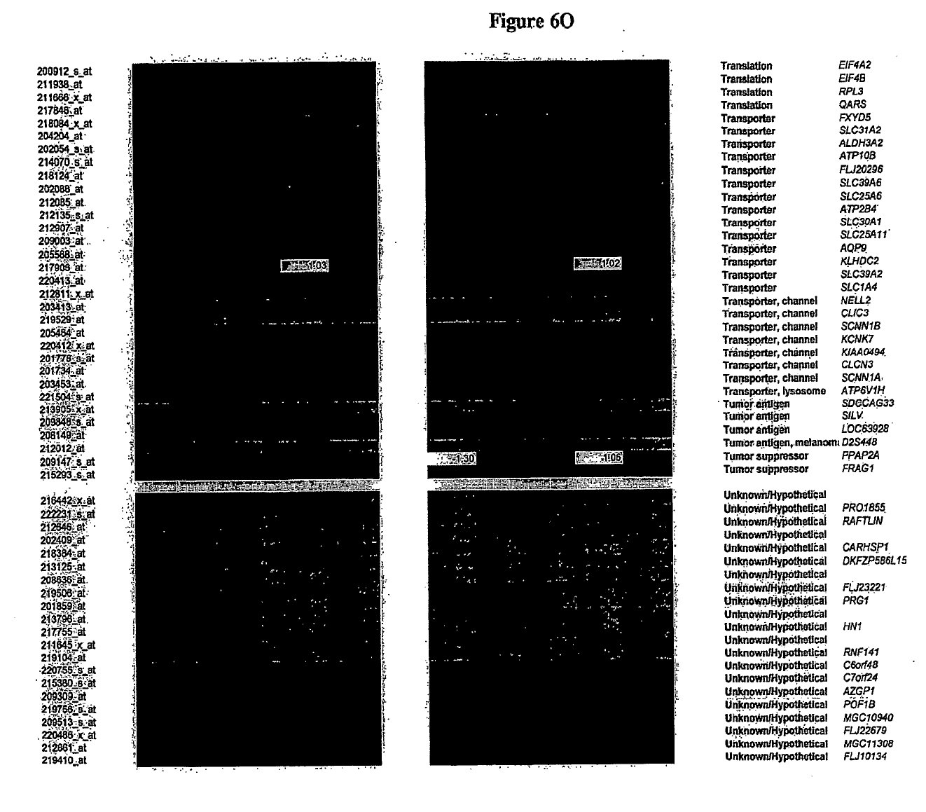

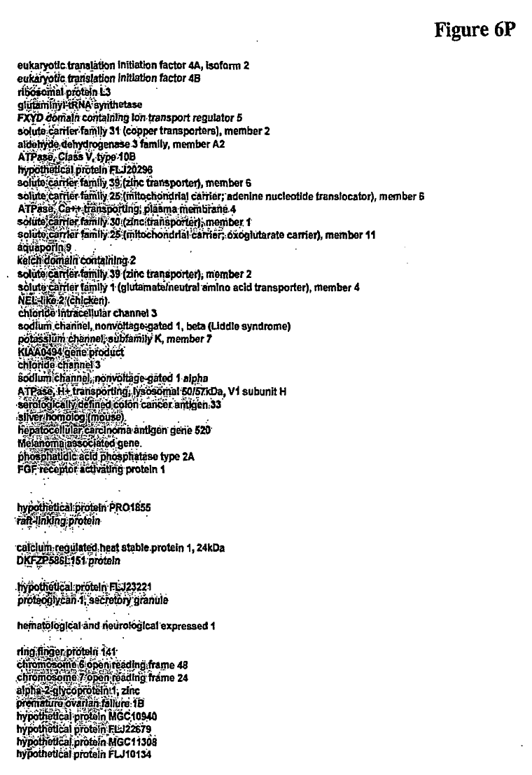

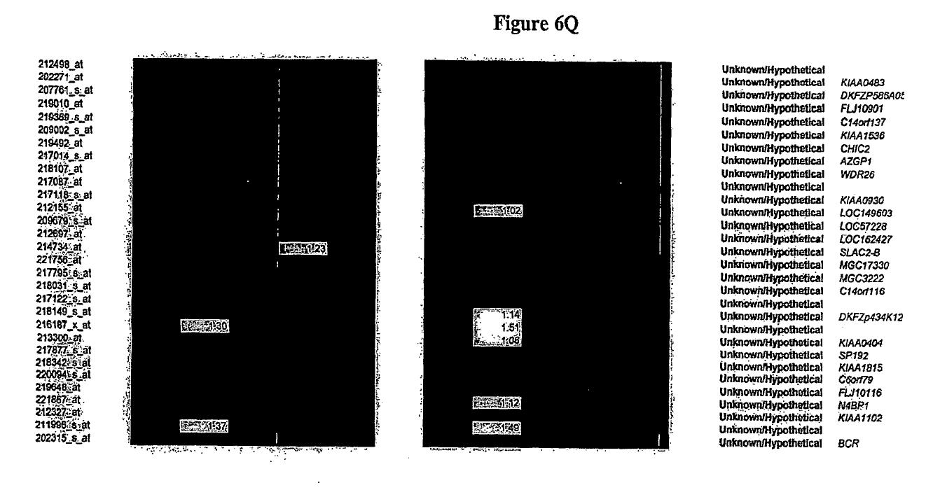

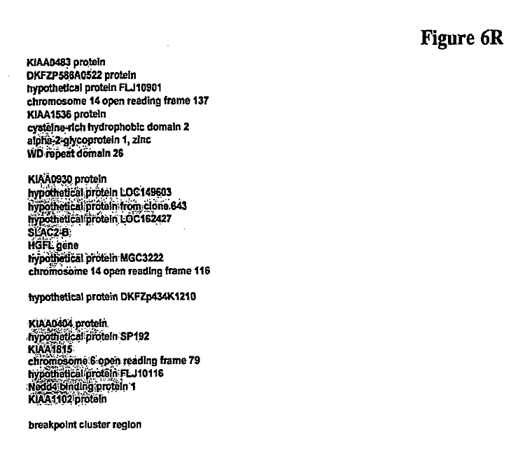

The results, found in FIG. 6, show the gene annotation table describing the molecular function and biological categories of the genes present on the Affymetrix Human Genome U133 GeneChip®. The light gray areas depict genes that are up-regulated in the tissue at location B, the non-ulcerated skin adjacent to the chronic wound (ACW) as compared to the tissue at location A, the non-healing edge of the wound (NHE). The dark gray areas depict genes that are down-regulated in tissues from location B as compared to location A. The numbers within the light gray and dark gray shaded areas depict the fold change. The two different columns depict the comparison of the two locations in two different patients. As seen by the Figure, over 400 genes are differentially regulated in the cells of the tissue in non-ulcerated skin adjacent to a chronic wound as compared to the cells of the tissue in the non-healing edge.

### Example 7

### Additional Skin Specimens and Histology

Additional skin sample biopsies were obtained from both the non-healing edge of chronic wounds (NHE) and normal healthy skin specimens. Skin biopsies from the non-healing edge of chronic wounds were obtained after surgical debridement procedures from three consenting patients with venous reflux ulcers. Three normal skin specimens were obtained as discarded tissue from voluntary corrective surgery.

A small portion of skin biopsies were embedded in OCT compound (Tissue Tek, Torrance, Calif.) and frozen in liquid nitrogen. The majority of the samples were stored in RNAlater (Ambion, Foster City, Calif.) for subsequent RNA isolation.

Hematoxylin and eosin staining were performed on tissue from the samples as described in Example 1. Similar to the results in Exhibit 1, all of the samples from the chronic wounds (NHE) showed hyperproliferative, hyper and para-keratotic epidermis typical for non-healing edges of chronic ulcers.

### Example 8

### Total RNA Isolation and Microarray Analysis of Additional Skin Samples

### Materials and Methods

Samples from Example 7 (three from the patients with the chronic wounds and three from normal skin), stored in RNAlater were used for RNA isolation and gene array data analysis, using the procedure described in Example 2.

### Results

Using the Affymetric HU133 chips and Gene Spring™ software previously described (Example 2), a gene tree utilizing all genes present on the chip, and a visualized expression profile of each sample were generated. This method allows overall visualization of the entire gene expression pattern, rather than specific gene regulation. As shown in FIG. 7 and previously described in Example 2, it is shown that the expression patterns of the skin samples from the chronic wound biopsies are similar, while the expression patterns of the samples taken from the normal skin samples are similar to each other but quite different from the pattern of the samples from the chronic wound.

### Example 9

### Further Analysis of Expression of Specific Genes of the Additional Skin Samples

### Materials and Methods

Using the samples from Example 7, further analysis of the actual genes being up-regulated and down-regulated in the gene expression profile obtained in Example 8 were done using the methods described previously in Example 6.

### Results

Of approximately 22,000 genes presented on the chip, 1557 genes were found to be differentially regulated between non-healing edges of the chronic wounds and normal healthy skin. Out of the 1557 genes, 55% of the genes were down-regulated and 45% were up-regulated in normal skin as compared to skin from the non-healing edges of a chronic wound. The regulated genes sorted by biological function and regulation are shown in Table 3.

TABLE 3
Percent of up-regulated and down-regulated genes in normal
skin as compared to skin from the non-healing edge of a
chronic wound

| PERCENT | | PERCENT |
|---|---|---|
| OF DOWN- | | OF UP- |
| REGULATED | BIOLOGICAL FUNCTION | REGULATED |
| GENES | OF THE GENES | GENES |
| 52 | Adhesion | 48 |
| 60 | Antioxidants | 40 |
| 53 | Apoptosis | 47 |
| 20 | Ca Binding | 80 |
| 47 | Cell cycle | 53 |
| 83 | Cell growth, proliferation | 17 |
| 75 | Cytochrome | 25 |
| 59 | Cytoskeletal | 41 |
| 29 | Detoxification | 71 |
| 80 | Development | 20 |
| 80 | DNA Binding | 20 |
| 45 | DNA Repair, Synthesis | 55 |
| 45 | ECM | 55 |
| 23 | Energy | 77 |
| 42 | Enzyme | 58 |
| 13 | Epidermal Differentiation | 87 |
| 47 | Golgi Apparatus | 53 |
| 54 | G-regulated Protein | 46 |
| 67 | Heat Shock | 33 |
| 47 | Immune Response Related | 53 |
| 8 | Immunoglobulin | 92 |
| 67 | INF-Regulated | 33 |
| 44 | Membrane Protein | 56 |
| 38 | Membrane, cell-surface | 62 |
| 38 | Metabolism | 62 |
| 27 | Mitochondrial | 73 |
| 86 | Nuclear Receptors | 14 |
| 54 | Nucleoskeletal | 46 |
| 67 | Oncogenesis | 33 |
| 46 | Proteolysis | 54 |
| 64 | Phosphatase | 36 |
| 80 | Protein Binding | 20 |
| 53 | Protein Kinase | 67 |
| 33 | Protein Inhibitor | 67 |
| 18 | Protein Modification | 82 |
| 58 | Receptors | 42 |
| 58 | Regulators | 42 |
| 85 | RNA Metabolism | 15 |
| 63 | Secreted | 37 |
| 63 | Signal Transduction | 37 |
| 58 | Trafficking | 42 |
| 70 | Transcription | 30 |
| 79 | Transcription Factor | 21 |
| 83 | Transcription Repressor | 17 |
| 70 | Translation | 30 |
| 47 | Transporter | 53 |
| 73 | Tumor Antigen | 27 |
| 57 | Tumor Suppressor | 43 |

The 100 most regulated genes, 50 being the most up-regulated and 50 being the most down-regulated, along with associated fold-changes and p-values, grouped by cellular functins and biological processes, are shown in FIG. 8. The most regulated genes fall into the following categories for biological processes: 1) contact and motility; 2) tissue remodeling; 3) inflammation; 4) proliferation; 5) differentiation; 6) cell death control; 7) metabolism; and 8) signal transduction and transcription.

### Example 10

### Immunohistochemistry

### Materials and Methods

In order to confirm the microarray data obtained in Example 9, the normal healthy skin samples and the skin samples from the chronic wounds (Example 7) were stained with antibodies recognizing various proteins that were differentially regulated in the chronic wound tissue.

Frozen skin specimens from both normal skin biopsies and biopsies from chronic wounds were cut with a cryostat (Jung Frigocut 28006, Leica, Germany) and stored at −80° C. Slides containing the frozen 5 micrometer skin sections were fixed in cold acetone for 1 minute. Sections stained with desmoglein 2 (1:2, AbCam, Cambridge, Mass.), desmoglein 3 (1:100, Santa Cruz Biotech, Santa Cruz, Calif.), and desmoplakin (1:200, a gift from Dr. Jim Wahl, University of Toledo) as a primary antibody were blocked with 0.1% Triton-X in 1% BSA for 60 minutes and incubated overnight at 4° C.

Sections stained with a monoclonal antibody against filaggrin (1:1000 as described in Dale et al. (1985) J. Cell. Biol. 101: 1257-1269), keratin 10 (1:500, a gift from Dr. Tung-Tien Sun, New York University School of Medicine), and involucrin (1:500, NeoMarkers, Waltham, Mass.) as a primary antibody were blocked with 5% bovine serum albumin (BSA) and incubated with a primary antibody diluted in 5% BSA in 1× phosphate buffered saline (PBS).

Signals were visualized using Alexa-Fluor 488 or Alexa-Fluor 594 (Molecular Probes, Carlsbad, Calif.) as a secondary antibody. Slides were mounted with mounting media containing Dapi (Vector Labs, Burlingame, Calif.).

All negative controls were prepared by substituting the primary antibody with PBS. Staining was analyzed using a Nikon Eclipse E800 microscope and digital images were collected using SPOT-Camera Advanced Program.

### Results

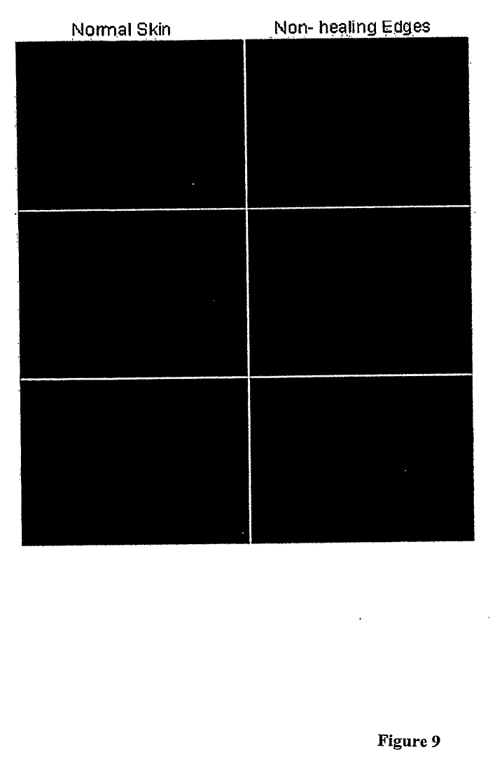

Desmoglein 2 (Dsg2), desmoglein 3 (Dsg3), and desmoplakin (DP) are adhesion junction molecules. Some adhesions junction molecules, including these three, were found to be differentially regulated in chronic wounds in the microarray analysis performed in Example 9. Specifically, the microarray analysis showed that Dsg3 was up-regulated in chronic non-healing wounds, and Dsg2 and DP were down-regulated. As shown in FIG. 9, staining with Dsg3 showed an increased signal throughout the epidermis of the chronic wounds as compared to normal skin, while the staining signal of the Dsg2 and DP was decreased in the epidermis of the chronic wound. These data confirm that there is deregulation of major desmosomal proteins in the epidermis of chronic non-healing wounds.

Microarray analysis also showed that keratinocyte differentiation markers were differentially regulated in the epidermis of chronic non-healing wounds. Keratin 10 (K10) was shown to be down-regulated in the epidermis of chronic non-healing wounds. Additional differentiation markers, such as filaggrin (FLG) were also down-regulated, while involucrin (IVL) was up-regulated. The results of the immunohistochemistry analysis confirm the microarray data. As shown in FIG. 10, there is an increased involucrin expression in the epidermis of the chronic non-healing wounds, whereas the K10 and filaggrin staining was barely detected in the chronic non-healing wound samples.

In conclusion, the results from the immunohistochemistry analysis confirm and are in agreement with the results from the microarray analysis.

### Example 11

### Quantitative Real-Time PCR Analysis

### Materials and Methods

0.5 μg of total RNA from normal skin samples and samples from the chronic wounds were reverse transcribed using Omniscript Reverse Transcription Kit (QIAGEN). The real-time PCR was performed in triplicate using the iCycler iQ thermal cycler and detection system and an iQ SYBR Supermix (BioRad, Hercules, Calif.). Relative expression was normalized for levels of hypoxanthin-guanine phosphoribosyltransferase (HPRT1). The primer sequences used were as follows:

| | |
|---|---|
| HPRT1, forward - | |
| (5′-AAAGGACCCCACGAAGTGTT-3′) | (SEQ ID NO 1) |
| HPRT1, reverse - | |
| (5′-TCAAGGGCATATCCTACAACAA-3′) | (SEQ ID NO 2) |

Human β defensin 4 (HBD4), forward -

| | |
|---|---|
| (5′-GGTGGTATAGGCGATCCTGTT-3′) | (SEQ ID NO 3) |
| HBD4, reverse - | |
| (5′-AGGGCAAAAGACTGGATGACA-3′) | (SEQ ID NO 4) |
| Kalikrein 6 (KLK6), forward - | |
| (5′CATGGCGGACCCCTGCGACAAGAC-3′) | (SEQ ID NO 5) |
| KLK6, reverse - | |
| (5′-TGGATCACAGCCCGGACAACAGAA-3′) | (SEQ ID NO 6) |
| MMP11, forward - | |
| (5′-AGATCTACTTCTTCCGAGGC-3′) | (SEQ ID NO 7) |
| MMP11, reverse - | |
| (5′-TTCCAGAGCCTTCACCTTCA-3′) | (SEQ ID NO 8) |
| CCL27-2, forward - | |
| (5′-TCCTGAGCCCAGACCCTAC-3′) | (SEQ ID NO 19) |
| CCL27-2, reverse - | |
| (5′-CAGTTCCACCTGGATGACCTT-3′) | (SEQ ID NO 10) |
| APOD, forward - | |
| (5′-AATCAAATCGAAGGTGAAGCCA-3′) | (SEQ ID NO 11) |
| APOD, reverse - | |
| (5′-ACGAGGGCATAGTTCTCATAGT-3′) | (SEQ ID NO 12) |
| S100A7, forward - | |
| (5′-GGAGGAACTTCCCCAACTTCC-3′) | (SEQ ID NO 13) |
| S100A7, reverse - | |
| (5′-ACATCGGCGAGGTAATTTGT-3′) | (SEQ ID NO 14) |
| BMP2, forward - | |
| (5′-TCAAGCCAAACACAAACAGC-3′) | (SEQ ID NO 15) |
| BMP2, reverse - | |
| (5′-GTGGCAGTAAAAGGCGTGAT-3′) | (SEQ ID NO 16) |
| BMP7, forward - | |
| (5′-AGGCCTGTAAGAAGCACGAG-3′) | (SEQ ID NO 17) |
| BMP7, reverse - | |
| (5′-GGTGGCGTTCATGTAGGAGT-3′) | (SEQ ID NO 18) |

Statistical comparisons of expression levels from the chronic wounds versus the normal skin were performed using the Student's t-test.

### Results

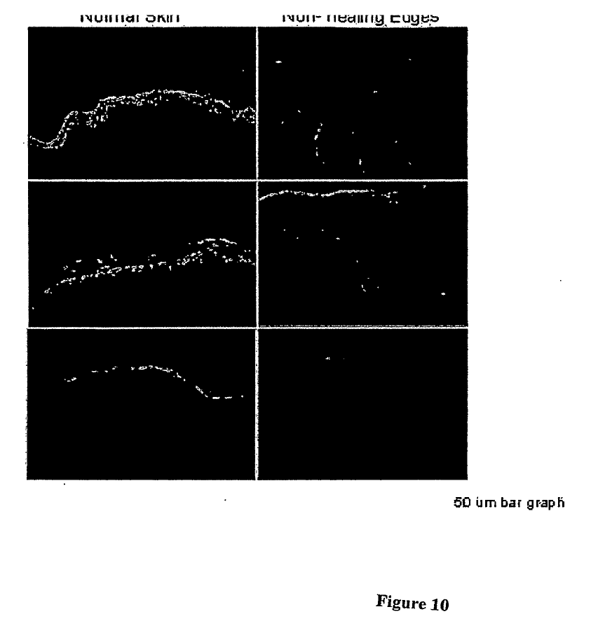

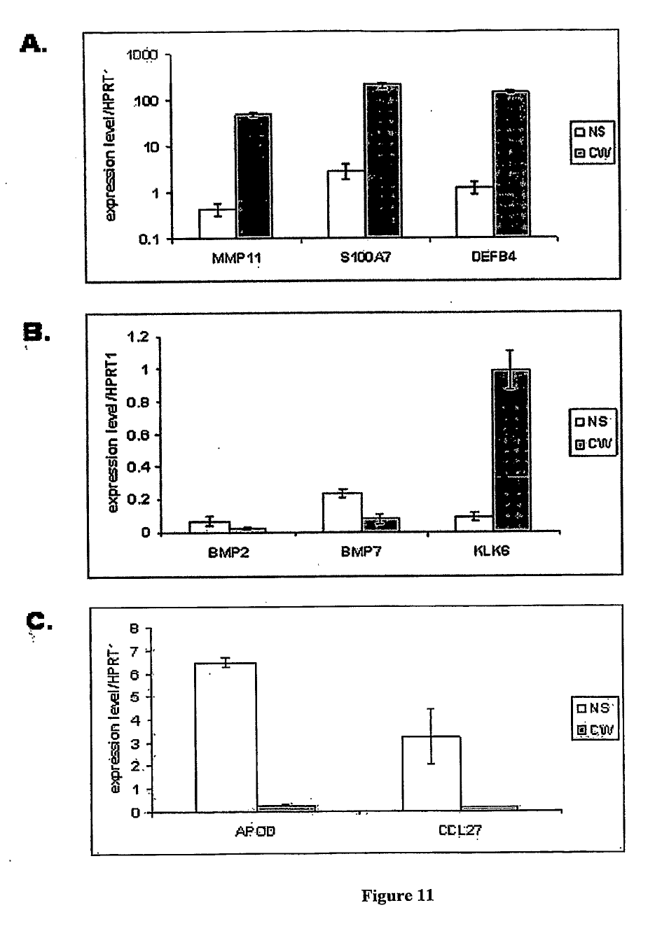

The results of the PCR analysis are shown in FIG. 11. S1007A, a gene which is part of the human epidermal differentiation complex (EDC) and belongs to the S100 family, was among the most 50 up-regulated genes in chronic wound epidermis as found by microarray analysis. As shown in FIG. 11(A), S1007A was expressed almost 100 fold in the chronic wound tissue. Additionally, as shown in FIG. 11(A), DEFB4, associated with benign hyperplasia in skin, was also expressed almost 100 fold more in the chronic wound epidermis as compared to the normal epidermis. This is consistent with the microarray analysis. Also, the expression of MMP-11 was greatly increased in the chronic wound tissue as compared to the normal skin as shown in FIG. 11(A). Again, this is consistent with the microarray analysis.

As shown in FIG. 11(B), bone morphogenetic proteins, BMP2 and BMP7, had much lower expression levels in the chronic wound skin. This is consistent with the microarray analysis which showed these genes to be among the 50 most down-regulated genes in chronic wound epidermis. Also shown in FIG. 11(B), RT-PCR analysis showed the expression levels of KLK6 is greatly increased in chronic wound epidermis. This protein has been implicated in keratinocyte proliferation and differentiation and in the pathogenesis of psoriasis.

FIG. 11(C) shows that the expression of both APOD and CCL27, cutaneous T cell attracting chemokin, are highly suppressed in the chronic non-healing wounds.

In conclusion, the RT-PCR analysis confirmed the results of the microarray analysis.

The present invention is not limited in scope by specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for the identification of a margin of debridement within or adjacent to a chronic wound suitable for debriding, comprising:
(a) obtaining a tissue sample from a site within or adjacent to the chronic wound;
(b) determining a gene expression profile of the tissue sample; and
(c) comparing the gene expression profile of the tissue sample with a known gene expression profile of tissue from a known site of non-ulcerated skin adjacent to the chronic wound,
wherein if the gene expression profile of the tissue sample is the same or similar to the known gene expression profile of the tissue from the known site, then the site of the tissue sample has reached the margin of debridement.

2.-5. (canceled)

6. The method of claim 1, wherein the gene expression profiles of the tissue sample and the tissue from the known site are determined by microarray analysis.

7. The method of claim 1, wherein the known gene expression profile for skin adjacent to the chronic wound is found in FIG. 2.

8. A method for determining whether a chronic wound is in further need of debriding, comprising:
(d) obtaining a tissue sample from within or adjacent to the chronic wound;
(e) determining a gene expression profile for the tissue sample;
(f) comparing the gene expression profile of the tissue sample with a known gene expression profile of tissue from a known site adjacent to the chronic wound,
wherein if the gene expression profile of the tissue sample is the same or similar to the known gene expression profile of the tissue from the known site, then the wound is not in need of further debriding.

9.-12. (canceled)

13. The method of claim 8, wherein the gene expression profiles of the tissue sample and the tissue from the known site are determined by microarray analysis.

14. The method of claim 8, wherein the known gene expression profile for skin adjacent to the chronic wound is found in FIG. 2.

15. The method of claim 8, wherein the tissue sample derives from tissue that has been previously debrided.

16. A method for the identification of a site within or adjacent to a chronic wound suitable for debriding, comprising:
(g) obtaining a tissue sample from a site within or adjacent to the chronic wound;
(h) determining the expression of a gene or genes known to be up-regulated or induced in tissue from chronic wounds; and
(i) comparing the expression of the gene or genes of the tissue sample with the known expression of up-regulated gene or genes from the chronic wound tissue;
wherein if the expression of the gene or genes of the tissue sample is identical or similar to the known expression of the gene or genes from the chronic wound tissue, then the site of the tissue sample is suitable for debriding, and if the expression of the gene or genes of the tissue sample is different from the known expression of the gene or genes from the chronic wound tissue, then the site of the tissue sample is not suitable for debriding.

17. The method of claim 16, wherein the genes that up-regulated or induced in the tissue from chronic wounds are desmocollin 2 (Dsc2), desmoglein 3 (Dsg3), involucrin (IVL), small proline rich protein 1A (SPRR1A), small proline rich protein 1B (SPRR1B), small proline rich protein 2B (SPRR2B), small proline rich protein 3 (SPRR3), transglutaminase 1 (TGM1), S100 calcium binding protein A7 (S100A7), S100 calcium binding protein A8 (S100A8), S100 calcium binding protein A9 (S100A9), manic fringe protein (MFNG), phospholipase D 2 (PLD2), kalikrein 6, (KLK6), septin (SEPT_{—}8), serine/threonine kinase 10 (STK10), serine/cysteine proteinase inhibitor, clade B, member 3 (SERPINB3), symplekin (SYMPK), cyclin B1, cyclin D2, cyclin A2, cyclin F, cyclin M4, cell division cycle 2 homolog (CDC2), cyclin dependent kinase inhibitor NB (CDKNB), cyclin dependent kinase inhibitor N3 (CDKN3), keratin 6 (K6), keratin 16 (K16), bone morphogenetic protein 1 (BMP-1), platelet derived endothelial growth factor (ECGF1), receptor neuropilin (NRP1), stromal cell derived factor 1-alpha (SDF-1α), defensin B4 (DEFB4), IL-7 receptor (IL-7R), platelet derived growth factor B (PDGFB), platelet derived growth factor A (PDGFA), Fas-activated serine/theorine kinase (FASTK), Fas (TNFRSF6) associated factor (FAF1), proapoptotic caspase adaptor protein (PCAP), bcl-2 associated protein (BAX), p53 inducible protein (TP5313), nucleolar protein 3(NOL3), apoptosis, caspase activation inhibitor (AVEN), and baculoviral IAP repeat-containing 5 (surivin) (BIRC5).

18. A method for the identification of a site within or adjacent to a chronic wound suitable for debriding, comprising:
j) obtaining a tissue sample from a site within or adjacent to the chronic wound;
(k) determining the expression of a gene or genes known to be down-regulated or suppressed in tissue from chronic wounds; and
(l) comparing the expression of the gene or genes of the tissue sample with the known expression of down-regulated gene or genes from the chronic wound tissue;
wherein if the expression of the gene or genes of the tissue sample is identical or similar to the known expression of the gene or genes from the chronic wound tissue, then the site of the tissue sample is suitable for debriding, and if the expression of the gene or genes of the tissue sample is different from the known expression of the gene or genes from the chronic wound tissue, then the site of the tissue sample is not suitable for debriding.

19. The method of claim 18, wherein the genes that down-regulated or suppressed in the tissue from chronic wounds are desmocollin 3 (Dsc3), desmoglein 2 (Dsg2), desmoplakin (DP), plakophilin 2 (PKP2), filaggrin (FLG), thrichohyalin (THH), kuppel-like factor (KLF4), NOTCH, drosophila, homolog OF, 2 (NOTH2), phospholipase D 1 (PLD1), protease inhibitor 3, skin-derived (SKALP, PI3), oxysterol binding protein-like 8 (OSBPL8), adducing 3 (ADD3), early growth response 3 (EGR3), inhibitor of DNA binding 4 (ID4), occluding (OCLN), decay accelerating factor for complement (DAF), tight junction protein, zona ocludens 3 (ZO3), tight junction protein 3 (TJP3), spectrin 1 (SPTBN1), multiple PDZ domain protein (MUPP1), InaD-like protein (INADL), claudin 5 (CLDN5), claudin 8 (CLDN8), protein kinase C-iota (PKC-iota), cell division cycle homolog 42 (CDC42), retinoblastoma protein (Rb), retinoblastoma protein (p107), retinoblastoma protein (p103), checkpoint suppressor (CHES1), WEE1 homolog (WEE1), translation initiation factor (EIF4E), insulin-like growth factor binding protein (IGFBP5), bone morphogenetic protein 2 (BMP-2), bone morphogenetic protein 7 (BMP-7), leptin receptor (LEPR), vascular endothelial growth factor (VEGF), epiregulin (EREG), angiopoetin-like 6 (ANGPTL6), apolipoprotein D (APOD), cutaneous T cell attracting chemokine 27 (CCL27), IL-7, transforming growth factor, beta 2 (TGFB2), transforming growth factor, beta 3, (TGFBR3), fibroblast growth factor 13 (FGF13), interleukin 6 (IL-6), pleckstrin homology-like domain, family A, member 2 (PHLDA2), programmed cell death (PDCD6), protein tyrosine phosphatase, non-receptor type 13 (APO-1/CD95 (Fas)-associated phophatase (PTPN13), apoptotic peptidase activating factor 1 (APAF1), and TNFα mediated apoptosis inhibitor (TNFAIP3).

20. A method for determining whether a chronic wound is in further need of debriding, comprising:
(m) obtaining a tissue sample from a site within or adjacent to the chronic wound;
(n) determining the expression of a gene or genes known to be up-regulated or induced in tissue from chronic wounds; and
(o) comparing the expression of the gene or genes of the tissue sample with the known expression of gene or genes from the chronic wound tissue;
wherein if the expression of the gene or genes of the tissue sample is identical or similar to the known expression of the gene or genes from the chronic wound tissue, then the site of the tissue sample is in need of further debriding, and if the expression of the gene or genes of the tissue sample is different from the known expression of the gene or genes from the chronic wound tissue, then the site of the tissue sample is not in need of further debriding.

21. The method of claim 20, wherein the genes that up-regulated or induced in the tissue from chronic wounds are desmocollin 2 (Dsc2), desmoglein 3 (Dsg3), involucrin (IVL), small proline rich protein 1A (SPRR1A), small proline rich protein 1B (SPRR1B), small proline rich protein 2B (SPRR2B), small proline rich protein 3 (SPRR3), transglutaminase 1 (TGM1), S100 calcium binding protein A7 (S100A7), S100 calcium binding protein A8 (S100A8), S100 calcium binding protein A9 (S100A9), manic fringe protein (MFNG), phospholipase D 2 (PLD2), kalikrein 6, (KLK6), septin (SEPT_{—}8), serine/threonine kinase 10 (STK10), serine/cysteine proteinase inhibitor, clade B, member 3 (SERPINB3), symplekin (SYMPK), cyclin B1, cyclin D2, cyclin A2, cyclin F, cyclin M4, cell division cycle 2 homolog (CDC2), cyclin dependent kinase inhibitor NB (CDKNB), cyclin dependent kinase inhibitor N3 (CDKN3), keratin 6 (K6), keratin 16 (K16), bone morphogenetic protein 1 (BMP-1), platelet derived endothelial growth factor (ECGF1), receptor neuropilin (NRP1), stromal cell derived factor 1-alpha (SDF-1α), defensin B4 (DEFB4), IL-7 receptor (IL-7R), platelet derived growth factor B (PDGFB), platelet derived growth factor A (PDGFA), Fas-activated serine/theorine kinase (FASTK), Fas (TNFRSF6) associated factor (FAF1), proapoptotic caspase adaptor protein (PCAP), bcl-2 associated protein (BAX), p53 inducible protein (TP5313), nucleolar protein 3(NOL3), apoptosis, caspase activation inhibitor (AVEN), and baculoviral IAP repeat-containing 5 (surivin) (BIRC5).

22. (canceled)

23. A method for determining whether a chronic wound is in further need of debriding, comprising:
(p) obtaining a tissue sample from a site within or adjacent to the chronic wound;
(q) determining the expression of a gene or genes known to be down-regulated or suppressed in tissue from chronic wounds; and
(r) comparing the expression of the gene or genes of the tissue sample with the known expression of gene or genes from the chronic wound tissue;
wherein if the expression of the gene or genes of the tissue sample is identical or similar to the known expression of the gene or genes from the chronic wound tissue, then the site of the tissue sample is in further need of debriding, and if the expression of the gene or genes of the tissue sample is different from the known expression of the gene or genes from the chronic wound tissue, then the site of the tissue sample is not in need of further debriding.

24. The method of claim 23, wherein the genes that down-regulated or suppressed in the tissue from chronic wounds are desmocollin 3 (Dsc3), desmoglein 2 (Dsg2), desmoplakin (DP), plakophilin 2 (PKP2), filaggrin (FLG), thrichohyalin (THH), kuppel-like factor (KLF4), NOTCH, drosophila, homolog OF, 2 (NOTH2), phospholipase D 1 (PLD1), protease inhibitor 3, skin-derived (SKALP, PI3), oxysterol binding protein-like 8 (OSBPL8), adducing 3 (ADD3), early growth response 3 (EGR3), inhibitor of DNA binding 4 (ID4), occluding (OCLN), decay accelerating factor for complement (DAF), tight junction protein, zona ocludens 3 (ZO3), tight junction protein 3 (TJP3), spectrin 1 (SPTBN1), multiple PDZ domain protein (MUPP1), InaD-like protein (INADL), claudin 5 (CLDN5), claudin 8 (CLDN8), protein kinase C-iota (PKC-iota), cell division cycle homolog 42 (CDC42), retinoblastoma protein (Rb), retinoblastoma protein (p107), retinoblastoma protein (p103), checkpoint suppressor (CHES1), WEE1 homolog (WEE1), translation initiation factor (EIF4E), insulin-like growth factor binding protein (IGFBP5), bone morphogenetic protein 2 (BMP-2), bone morphogenetic protein 7 (BMP-7), leptin receptor (LEPR), vascular endothelial growth factor (VEGF), epiregulin (EREG), angiopoetin-like 6 (ANGPTL6), apolipoprotein D (APOD), cutaneous T cell attracting chemokine 27 (CCL27), IL-7, transforming growth factor, beta 2 (TGFB2), transforming growth factor, beta 3, (TGFBR3), fibroblast growth factor 13 (FGF13), interleukin 6 (IL-6), pleckstrin homology-like domain, family A, member 2 (PHLDA2), programmed cell death (PDCD6), protein tyrosine phosphatase, non-receptor type 13 (APO-1/CD95 (Fas)-associated phophatase (PTPN13), apoptotic peptidase activating factor 1 (APAF1), and TNFα mediated apoptosis inhibitor (TNFAIP3).

25. (canceled)

26. A method for the identification of a site within or adjacent to a chronic wound suitable for testing wound-healing therapeutic agents, comprising:
(s) obtaining a tissue sample from a site within or adjacent to the chronic wound;
(t) determining a gene expression profile of the tissue sample; and
(u) comparing the gene expression profile of the tissue sample with a known gene expression profile of tissue from a known site of non-ulcerated skin adjacent to the chronic wound;
wherein if the gene expression profile of the tissue sample is the same or similar to the known gene expression profile of the tissue from the known site, then the site of the tissue sample is suitable for testing wound-healing therapeutic agents.

27. The method of claim 26, wherein the tissue from the known site contains cells with healthy, normal morphology.

28. (canceled)

29. The method of claim 26, wherein the tissue from the known site contains cells that respond well to wound healing stimuli.

30. (canceled)

31. The method of claim 26, wherein the gene expression profiles of the tissue sample and the tissue from the known site are determined by microarray analysis.

32. The method of claim 26 wherein the known gene expression profile for skin adjacent to the chronic wound is found in FIG. 2.

33. The gene expression profile for skin adjacent to the chronic wound found in FIG. 2.

34. The gene expression profile for normal healthy skin found in FIG. 7.

35. The gene expression profile for skin from the non-healing edge of a chronic wound found in FIGS. 2 and 7.


SEQUENCE LISTING (18 sequences)

SEQ ID NO: 1 - DNA, 20 bp, Artificial Sequence
  primer
aaaggaccccacgaagtgtt

SEQ ID NO: 2 - DNA, 22 bp, Artificial Sequence
  primer
tcaagggcatatcctacaacaa

SEQ ID NO: 3 - DNA, 21 bp, Artificial Sequence
  primer
ggtggtataggcgatcctgtt

SEQ ID NO: 4 - DNA, 21 bp, Artificial Sequence
  primer
agggcaaaagactggatgaca

SEQ ID NO: 5 - DNA, 24 bp, Artificial Sequence
  primer
catggcggacccctgcgacaagac

SEQ ID NO: 6 - DNA, 24 bp, Artificial Sequence
  primer
tggatcacagcccggacaacagaa

SEQ ID NO: 7 - DNA, 20 bp, Artificial Sequence
  primer
agatctacttcttccgaggc

SEQ ID NO: 8 - DNA, 20 bp, Artificial Sequence
  primer
ttccagagccttcaccttca

SEQ ID NO: 9 - DNA, 19 bp, Artificial Sequence
  primer
tcctgagcccagaccctac

SEQ ID NO: 10 - DNA, 21 bp, Artificial Sequence
  primer
cagttccacctggatgacctt

SEQ ID NO: 11 - DNA, 22 bp, Artificial Sequence
  primer
aatcaaatcgaaggtgaagcca

SEQ ID NO: 12 - DNA, 22 bp, Artificial Sequence
  primer
acgagggcatagttctcatagt

SEQ ID NO: 13 - DNA, 21 bp, Artificial Sequence
  primer
ggaggaacttccccaacttcc

SEQ ID NO: 14 - DNA, 20 bp, Artificial Sequence
  primer
acatcggcgaggtaatttgt

SEQ ID NO: 15 - DNA, 20 bp, Artificial Sequence
  primer
tcaagccaaacacaaacagc

SEQ ID NO: 16 - DNA, 20 bp, Artificial Sequence
  primer
gtggcagtaaaaggcgtgat

SEQ ID NO: 17 - DNA, 20 bp, Artificial Sequence
  primer
aggcctgtaagaagcacgag

SEQ ID NO: 18 - DNA, 20 bp, Artificial Sequence
  primer
ggtggcgttcatgtaggagt